(19) United States
(12) Patent Application Publication
(10) Pub. No.: US 2012/0071901 A1
(43) Pub. Date: Mar. 22, 2012

(54) METHODS AND DEVICES FOR DELIVERING SUTURES IN TISSUE

(75) Inventor: Scott H. HENEVELD, Whitmore, CA (US)

(73) Assignee: Spirx Closure, LLC, Whitmore, CA (US)

(21) Appl. No.: 13/092,047

(22) Filed: Apr. 21, 2011

Related U.S. Application Data
(63) Continuation of application No. PCT/US2009/061712, filed on Oct. 22, 2009.
Application No. PCT/US2009/061712 is a continuation-in-part of application No. PCT/US2009/051442, filed on Jul. 22, 2009.
(60) Provisional application No. 61/107,622, filed on Oct. 22, 2008.

(51) Int. Cl. A61B 17/04

(52) U.S. Cl. 606/145; 606/144

(57) ABSTRACT

Methods and devices for driving a suture assembly employing elastically pre-shaped needles for piercing a tissue. The pre-shaped needles are held in a constrained state and can revert to a natural pre-shaped state prior to or during ejection from the device before entry into tissue allowing for the suture to follow a defined path similar to the pre-shaped needle such that removal of the needle allows for subsequent securing of the suture in or around tissue.

### CROSS-REFERENCE TO RELATED APPLICATIONS

This application is a continuation of International Application No. PCT/US2009/061712 filed Oct. 22, 2009, which claims benefit of priority to U.S. Provisional Application No. 61/107,622 filed Oct. 22, 2008; International Application No. PCT/US2009/061712 is also a continuation-in-pan of International Application No. PCT/US2009/051442 filed Jul. 22, 2009, and entitled “Methods and Devices for Delivering Suture in Tissue”; the entirety of which are incorporated by reference herein.

### BACKGROUND OF THE INVENTION

The present invention relates generally to systems and methods for the driving of a needle or suture through or into body tissue (typically, the needle will be affixed to a suture that remains in the tissue) using a catheter, introducer or other minimally invasive means. The methods and devices described herein can be used in any number of medical procedures, including but not limited to, approximating tissue (e.g., bring separated tissue together), ligating tissue (e.g., encircling or tying off), and fixating of tissue (attaching tissue to another structure or different tissue).

Commonly known suture systems mechanically drive needles thru the tissue wall to create passage for a suture. Such mechanisms are often complicated and require a skilled operator. In addition, the conventional mechanisms can involve many procedural steps to manipulate a needle to conform to a path to properly position the suture into tissue.

Conventional suture driving systems used for wound closure provides one example of existing suture driving systems. Such wound closure systems are used in transluminal medical procedures that are seeing a rise in popularity due to the reduction in surgical damage to healthy tissue, decreased recovery time, and ultimate cost savings to the patient associated with these procedures.

These transluminal procedures typically require a puncture into a body lumen and through the overlying tissue for the passing of catheters, guide wires, laparoscopes, endoscopes, vascular devices, etc. as required by the particular procedure. The punctures are created with instruments such as access needles, trocar, introducer sheaths, or other access devices and may measure from 1 to upwards of 15 mm in diameter. After completion of the procedure, the physician can utilize a closure system to close the puncture quickly to prevent further bleeding.

Manual compression of arterial or venous punctures is a common closure technique and an alternative to such closure systems. In this closure technique, medical personnel apply continuous pressure to the wound site allowing the blood to eventually clot sufficiently sealing the wound. However, this technique is typically very time consuming, requires the patient to bedridden for an extended time, and is not applicable for punctures over 4 mm. The longer recovery time increases overall cost and decreases patient satisfaction.

Sutures remain the preferred method of sealing such wounds, but the limited access and small size of the typical wound formed during a transluminal procedure complicates the task of sealing these wounds.

Generally, a physician must introduce a suture needle through the tissue tract and into the body lumen, position the needle, then passed the needle through tissue pulling the suture through as well. A number of devices are disclosed in U.S. Pat. No. 5,374,275 to Bradley et al., U.S. Pat. No. 5,364,408 to Gordon, U.S. Pat. No. 5,320,632 to Heidmueller, U.S. Pat. No. 5,403,329 to Hinchcliffe, U.S. Pat. No. 5,368,601 to Sauer et al., U.S. Pat. No. 5,431,666 to Auer et al. and international publications WO 94/13211 and WO 95/13021 each of the above references is incorporated by reference herein.

While these devices allow for sealing of the wound and driving the suture and needle through tissue, they are relatively complex and employ a significant number of moving parts. Accordingly, these devices are relatively costly to produce and are prone to mechanical failure.

U.S. Pat. Nos. 5,527,322, 5,792,152, 6,206,893, and 6,517,553 all to Klein U.S. and U.S. Pat. No. 5,972,005 to Stalker (each of the above is incorporated by reference herein) describes devices employing flexible or pre-shaped curved needles that are deformed from a natural shape during insertion or during advancement in tissue to close a puncture wound. U.S. Pat. No. 7,377,926 to Topper et al. (incorporated by reference herein) teaches another system for inserting a needle. In this variation, the insertion device houses a bendable needle in one of the jaws and is adapted to carry a suture

However, systems, such as those described above often deform a needle to drive a suture. Deformation of the needle in this manner often results in device malfunction when placing the suture, or requires significant additional complex components to ensure proper movement of the needle and suture as desired. Accordingly, there remains a need for a simple mechanized device and method to accurately and precisely drive a suture through tissue in a constrained space such as is required in less invasive procedures.

Such systems can also perform closure of openings in organs, whether to repair a defect, to close a wound, or to close an incision made in the organ for the purpose of accessing the organ to perform a separate medical procedure. As one example, when performing valve repair or replacement within the heart, a surgeon can access an apex of the heart after performing a thoracotomy or a mini-thoracotomy. The thoracotomy allows the surgeon to manually close the opening in the heart tissue via a suture pattern. Such a pattern can include one or more concentric purse string suture patterns to ensure closure of the opening into the heart. Percutaneous access to the heart to perform such valve procedures results in many of the same benefits as other percutaneous procedures. Namely, reduced complications, cost and recovery time on the part of the patient. However, percutaneous access leaves the surgeon with a small access path to close the opening in the heart.

The anatomical structure of the apical area permits the introduction of various surgical devices and tools into the heart without significant disruption of the natural mechanical and electrical heart function. Access to the heart through the femoral vessels in percutaneous methods is limited to the diameter of the vessel (approximately 8 mm). However, access to the heart through the apical area allows for a significantly larger access path (approximately 25 mm). Thus, apical access to the heart permits greater flexibility with respect to the types of devices and surgical methods that may be performed in the heart and great vessels. Such access is disclosed in Bergheim, US Patent Application 20050240200, the entirety of which is incorporated by reference. Accordingly, there remains a need for a simple mechanized device that can accurately and precisely drive a suture through tissue allows the surgeon to close the heart tissue and complete the procedure in a percutaneous manner.

In addition, the methods and systems described herein have additional uses other than closure of tissue. In another example, U.S. Patent Application No. 20070203479 to Auth et al. (incorporated by reference herein) describes methods and devices, and systems for the partial or complete closure or occlusion of a patent foramen ovale (“PFO”). An improved suture driving device can be used for fixating tissue and eliminate the need for such implantable devices.

Accordingly, the need continues to exist for an improved suturing systems and methods that drive a suture for approximating tissue, ligating tissue, and/or fixating of tissue.

### SUMMARY OF THE INVENTION

The following description includes an example of the methods and devices within the scope of this disclosure. It is also contemplated that combinations of aspects of various embodiments as well as the combination of the various embodiments themselves is within the scope of this disclosure.

In one variation, the invention includes a suture driving assembly for positioning a suture in a tissue section, the assembly comprising at least one needle assembly having a tissue piercing end distal to an elongate shaped section, the elongate shaped section having a curvilinear shape, the elongate shaped section being elastically deformable when restrained into a strained state and upon release assumes the curvilinear shape, the suture coupled to the needle assembly; a main body having a tissue engaging surface at a distal end, at least one constraining channel and at least one retrieving channel each of which having an opening at the tissue engaging surface; such that when the elongate shaped section of the needle assembly is in the restraining portion, the elongate shaped section is deformed into the strained state and when the elongate shaped section advances through the guide segment portion, the elongate shaped section assumes the curvilinear shape, upon continued advancement the elongated shaped section exits through the opening of constraining channel in the curvilinear shape; a suture retriever assembly located in the needle receiving channel.

Suture driving assemblies according to the present disclosure can include needle assemblies having elongate shaped sections comprising a cross sectional shape that has two axis that are not of equal length. In one example, such a cross sectional shape includes a non-circular cross-sectional shape. In such cases, the non-circular shaped needle assemblies are constructed to promote assuming the desired curvilinear shape. For example, the largest dimension of such a needle assembly will promote stability along that dimension so that bending occurs in line with the smallest dimension (e.g., bending will occur about the axis of the longest dimension). For example, if the needle assembly is rectangular, the bending of the needle shall occur in line with the smallest dimension of the cross-sectional shape. In additional variations, the suture driving assembly further comprises a constraining channel having at least a portion with a non-circular cross-sectional shape to allow a sliding fit with the non-circular cross-sectional shape of the elongate section of the needle assembly.

The suture driving assemblies described herein can optionally include a clamping member axially moveable relative to the tissue engaging surface. Optionally, the clamp member can be expandable with a first reduced profile and an expanded profile, where in the reduced profile the expandable member can advance through an opening in the tissue section and where the expandable member can be withdrawn toward the tissue supporting face to secure the tissue section therebetween. The clamping member may be displaced prior to the suture driving assembly being deployed or it may maintain its position, securing the tissue while the suture driving assembly is being deployed.

The needle assembly as well as the number of needle assemblies can vary depending upon the type of suture stitch required. For example, the device can include a single needle assembly having a single shaped section or multiple shaped sections. In alternate variations, the assembly comprises two or more needle assemblies. The needle assemblies as well as the shaped portions used in any particular suture driving mechanism need not have the same shape. Instead, a single suture driving assembly can use needle assemblies of differing shapes at the same time. However, the spacing and relation of the constraining channel and the retrieval channel shall be adjusted to accommodate a particular shape and configuration of a particular needle assembly.

In certain variations, the constraining channel can include a first cross-sectional shape and the guide segment has a second cross sectional shape, where the first and second cross-sectional shapes are different, where the second cross sectional shape permits at least a part of the shaped section of the needle assembly entering the guide segment to revert to the curvilinear shape prior to entry into the tissue.

In additional variations, the constraining channel can include a cross-sectional shape that corresponds to the cross-sectional shape of the respective needle deployed therein. This provides orientation between the constraining channel and the shaped section of the needle. When the elongate shaped section of the needle exits the distal end of the constraining channel, the needle travels in a path defined by its curvilinear shape. The constraining channel can be parallel to, or at an angle to, the primary longitudinal axis of the device.

The sutures used in the devices and methods described herein can include a needle assembly comprising a needle lumen extending through at least the tissue piercing end and where the suture is removably nested within the needle lumen. In additional variations, the suture can be located exterior to the needle assembly so that a first free end of the suture is inserted into the needle lumen at the tissue piercing end. In another variation, a single suture can be affixed at both ends to a needle assembly where the needle assembly comprises two shaped sections with each having a tissue piercing end.

Sutures used in the present devices and methods can be front loaded into a needle assembly. As a result, a suture retriever assembly can remove the suture from the needle assembly via a front portion of the needle assembly. In one example, the suture retriever assembly comprises at least one pawl member that reduces an opening of the retrieving channel to less than a size of the needle assembly and suture, where the pawl member is biased to allow movement of the needle assembly and suture in a first direction and resist movement of the needle assembly and suture in a second direction, where rearward movement of the needle assembly from the retrieving channel causes the paw member to compress and retain the suture within the retrieving channel.

Alternate suture retriever assembly can include structures selected from the group consisting of a set of jaws, a recessed notch, pawl, funnel, catch cloth, magnetic coupling device, finger trap, or other gripping mechanism.

The devices of the present disclosure can include one or more vacuum lumens at the tissue engaging surface for securing tissue thereagainst. Alternately, or in combination, the tissue engaging surface can include a bonding agent for securing tissue thereagainst.

The devices described herein can be combined with various other medical implements to aid in the closure of tissue. For example, the devices can include one or more pledgets that removably positioned on the tissue engaging surface for placement at the opening in tissue.

In another variation, a suture driving assembly for closing an opening in a tissue section can include a first needle assembly having a tissue piercing end distal and being elastically deformable when restrained into a strained state and upon release assumes the curvilinear shape; a suture exterior to the needle assembly and having at least one end front-loaded into a needle lumen of a first tissue piercing portion of the first needle assembly; a main body having a tissue engaging surface at a distal end, at least one constraining channel and at least one retrieving channel each of which having an opening at the tissue engaging surface; where the constraining channel extends through the main body and comprises at least a restraining portion having a profile to maintain the needle assembly into the strained state and a guide segment portion adjacent to the constraining channel opening and having a profile to release needle assembly into the curvilinear shape when advanced therethrough and upon continued advancement the needle assembly exits the opening of the constraining channel in the curvilinear shape; a suture retriever assembly located in the needle retrieving channel and comprising a pawl mechanism, where the pawl mechanism interferes with the front loaded suture and needle assembly when advanced therein, where rearward movement of the front loaded suture and needle assembly causes the pawl to engage the suture to retain the suture within the needle retrieving channel; and an expandable member axially moveable relative to the tissue engaging surface, the expandable member having a first reduced profile and an expanded profile, where in the reduced profile the expandable member can advance through an opening in the tissue section and where the expandable member can be withdrawn toward the tissue supporting face to secure the tissue section therebetween when expanded.

The present disclosure also includes methods positioning a suture in a wall of an organ to close an opening in the wall. In one variation, the method includes placing a main body adjacent to a proximal side of the tissue, where the main body comprises at least one needle assembly coupleable to the suture and within a constraining channel located in the main body, where the needle assembly comprises a tissue piercing end distal to an elongate shaped section, the elongate shape section having a curvilinear shape, the shaped section being elastically deformable into a strained state within the constraining channel, and a suture coupled to the needle assembly, the main body further including a tissue engaging surface; advancing a clamping member through the opening in the organ when the clamping member, if expandable, is in a reduced profile; expanding the clamping member to an expandable profile; positioning the wall of the organ between the main body and the clamping member by retracting the clamping member; advancing the needle assembly from the constraining channel whereupon it reverts to the curvilinear shape upon exiting from the constraining channel and entering the wall of the organ; driving the needle assembly through a proximal side of the wall of the organ, such that the shaped section moves through the curvilinear shape so that the tissue piercing distal end and suture re-enter the main body at a retrieving channel; fully reducing the clamping member if it is expandable into a reduced profile; and withdrawing the main body.

In another variation, the method may further include vacuum lumens at the tissue contacting surface to secure the tissue. After sufficient vacuum has been applied to stabilize and secure the tissue, the expandable member may be reduced in profile and/or displaced prior to advancing the needle assembly. After the suture has been passed through the tissue and the needle assembly retracted, the vacuum is released. The main body is then withdrawn.

In another variation, the method may further include advancing a plurality of needle assembly pairs, where each needle assembly pair is coupled to an end of a suture and where each needle assembly advances from a respective constraining channel into a respective guide segment, where the guide segment permits the shaped section of the respective needle assembly located therein to revert to the curvilinear shape prior to leaving the respective guide segment and enter the wall of the organ; and where the plurality of needle assemblies move through the curvilinear shape so that the tissue piercing distal end of each needle assembly pair re-enter the main body at a respective retrieving channel.

As described above, the method optionally includes the use of front-loaded sutures. Such sutures allow for securing the suture in the retrieving channel by advancing the needle assembly and suture against a pawl mechanism such that the pawl mechanism compresses the suture to retain the suture while allowing the needle assembly to be withdrawn back into the constraining channel.

The methods can include positioning the wall of the organ between the main body and the expandable member by axially moving the expandable member relative to the tissue engaging surface to capture the wall of the organ therebetween. To further stabilize the device, the method can include partially reducing the expandable member and positioning the partially reduced expandable member into the opening.

In another variation, the methods can include positioning a suture within a tissue of the heart for closing an opening in the heart. Such method can comprise placing a main body adjacent to an exterior surface of the heart, where the main body comprises at least one needle assembly coupled to the suture and within a constraining channel located in the main body, where the needle assembly comprises a tissue piercing end distal to an elongate shaped section, the elongate shape section having a curvilinear shape, the shaped section being elastically deformable into a strained state within the constraining channel, and a suture coupled to the needle assembly, the main body further including a tissue engaging surface; advancing an expandable member through the opening and into the heart when the expandable member is in a reduced profile; expanding the expandable member to an expandable profile; positioning the a portion of the heart between the main body and the expandable member; advancing the needle assembly from the constraining channel into a guide segment, where the guide segment permits the shaped section of the needle assembly located therein to revert to the curvilinear shape prior to leaving the guide segment and entering the wall of the organ; driving the needle assembly through a proximal side of the wall of the organ, such that the shaped section moves through the curvilinear shape so that the tissue piercing distal end and suture re-enter the main body at a retrieving channel; reducing the expandable member into a reduced profile; and withdrawing the main body.

In yet another variation, the device includes a suture driving assembly for positioning a suture in a tissue section having at least one needle assembly having a tissue piercing end distal to an elongate shaped section, the elongate shaped section having a curvilinear shape and a non-circular cross-sectional shape, the elongate shaped section being elastically deformable when restrained into a strained state and upon release assumes the curvilinear shape, the suture coupled to the needle assembly; a main body having a tissue engaging surface at a distal end, at least one constraining channel and at least one retrieving channel each of which having an opening at the tissue engaging surface; such that the constraining channel having a non-circular cross-sectional shape that corresponds to the non-circular cross-sectional shape of the respective elongate shaped section deployed therein, wherein an orientation between the constraining channel and the shaped section of the needle is provided, such that upon advancement the elongate shaped section of the needle exits the distal end of the constraining channel, the needle travels in a path defined by its curvilinear shape and the cross-sectional shape of the respective constraining channel and needle assembly; a suture retriever assembly located in the needle retrieving channel; a clamping member axially moveable relative to the tissue engaging surface, the clamping member having a first profile configured to advance through an opening in the tissue section and where the clamping member can be withdrawn toward the tissue supporting face to secure the tissue section therebetween.

In certain variations, the suture driving assembly can be used to drive a needle without any suture. In such a case, the needle can pick up the suture at the needle receiving passage, and upon retrieval, transport the suture through the tissue to re-enter the main body. An alternative method is the needle may be left within the tissue (to be removed later, to be absorbed by the native tissue, or for permanent placement.) Accordingly, needle driving assemblies having the same or similar structures disclosed herein are within the scope of this disclosure.

Additional suture driving assemblies are described in U.S. patent application Ser. No. 12/188,430 entitled METHODS AND DEVICES FOR DELIVERING SUTURES IN TISSUE filed on Aug. 8, 2008, the entirety of which is incorporated by reference.

### BRIEF DESCRIPTION OF THE DRAWINGS

FIG. 1A shows an example of a suture driving assembly.

FIG. 2A shows an isometric view of one variation of a needle or needle assembly with a back loaded suture.

FIG. 2B shows a side view of the needle assembly of FIG. 2A.

FIGS. 2C to 2F show variations of front loaded sutures.

FIG. 2G shows a variation of a needle assembly having two curved sections affixed to a single suture.

FIG. 3A illustrates a partial cross sectional view of a distal portion of a suture driving assembly.

FIG. 3B shows the distal portion of a suture driving assembly with the needle assembly advanced through guide segments of constraining channels within the main body.

FIGS. 4A to 4E illustrate a needle assembly advancing at a working surface of a main body of a suture driving assembly.

FIGS. 5A and 5B illustrate the needle assembly and suture respectively after actuation of the assembly.

FIGS. 5C and 5D illustrate an example of a laced suture driven by a needle assembly after passing through tissue about an opening in the tissue.

FIGS. 6A to 6C provide another example of an alternate configuration of a main body having two pairs of constraining and retrieval channels to produce a desired stitch

FIGS. 7A and 7B illustrate examples of needle or suture retrieval devices.

FIGS. 8A to 8D illustrate an example of a suture retrieval device used to retrieve a front-loaded suture.

FIGS. 8E to 8G illustrate another example of a suture retrieval device used to retrieve a front-loaded suture.

FIG. 9A shows another example of a suture driving assembly having a device that extends within a main body of the assembly along with a dilation device.

FIG. 9B illustrates the suture driving assembly of FIG. 9A

FIG. 9C shows an example of a dilation device.

FIG. 9D illustrates a front view of the suture driving assembly of FIG. 9B.

FIG. 9E is a perspective view of a tissue engaging surface of s suture driving assembly where the needle assemblies and sutures are extended from the constraining channels in the main body.

FIG. 9F illustrates an introducer and dilation device that can be advanced through the suture driving assembly of FIG. 9A.

FIGS. 10A to 10F illustrate an example of a suture driving assembly when used to place a suture in an organ. In the present example the suture is used to temporarily secure an access sheath for performing an additional procedure within the heart.

FIG. 11A shows an example of a suture driving assembly placed on a blood vessel.

FIG. 11B shows the suture pattern created by the suture driving assembly.

FIG. 12 illustrates a detail view revealing the living hinges of the expandable clamping member.

FIG. 13A illustrates the suture driving assembly with the clamping member entered into the vessel.

FIG. 13B illustrates the suture driving assembly with the clamping member in the expanded state.

FIG. 13B illustrates the suture driving assembly with the clamping member retracted to capture the vessel wall.

FIG. 13C illustrates the suture driving assembly with the needle assembly advanced thru the guide segments of the constraining channels within the main body.

FIG. 14A shows an example of a suture driving assembly placed on a blood vessel.

FIG. 14B shows the suture pattern created by the suture driving assembly

FIG. 14C illustrates a needle assembly advanced at working surface of a main body of a suture driving assembly.

FIGS. 15A to 15B show the needle assembly with rectangular cross-section needles.

FIG. 16 illustrates a cross sectional shape having a horizontal dimension that is longer than a vertical dimension, (e.g., a non-circular cross sectional shaped needle assembly) located within a corresponding non-circular cross shaped section of a constraining channel.

### DESCRIPTION OF THE PREFERRED EMBODIMENT

The above variations are intended to demonstrate the various examples of embodiments of the methods and devices of the invention. It is understood that the embodiments described above may be combined or the aspects of the embodiments may be combined in the claims.

The present invention relates generally to systems and methods for the driving of a needle or suture through or into body tissue (typically, the needle will be affixed to a suture that remains in the tissue) using a catheter, introducer or other minimally invasive means. The methods and devices described herein can be used in any number of medical procedures, including but not limited to, approximating tissue (e.g., bring separated tissue together), ligating tissue (e.g., encircling or tying off), and fixating of tissue (attaching tissue to another structure or different tissue).

As noted herein, the suture driving methods and assemblies described are discussed in relation to vascular wound closure allowing a physician to quickly, easily, and accurately insert a suture immediately following the procedure to prevent excessive blood loss by the patient. In addition the suture driving methods and devices can be used in various other areas (such as cardiology, urology, gynecology, or other vascular surgery applications) to approximate, ligate, or fixate tissue.

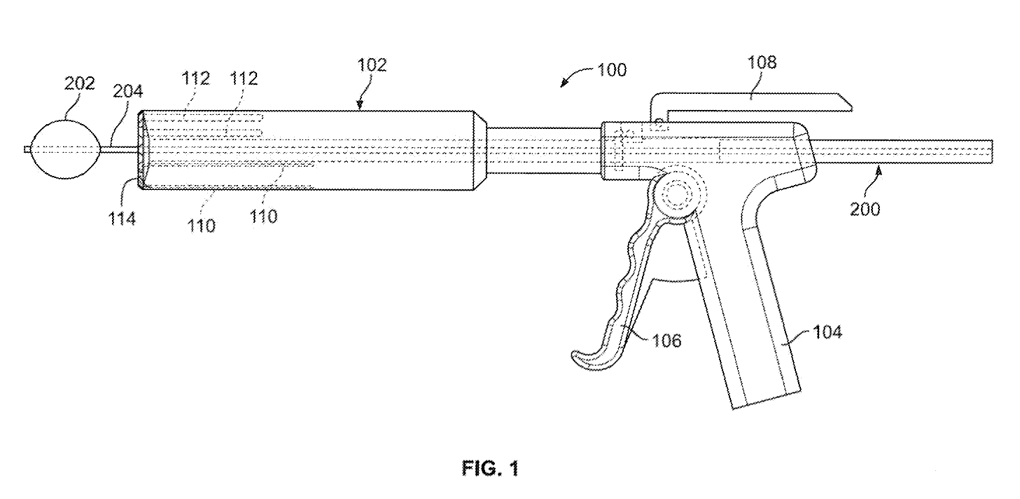

FIG. 1A shows an example of a suture driving assembly 100. In this variation, the suture driving assembly 100 includes a main body 102 coupled to a handle portion 104. The handle portion 104 can include any number of actuating triggers or levers 106, 108 where each lever functions to drive and/or retract the needle assembly to or from tissue. In the illustrated example, the needles are driven through the tissue or “thrown” via a trigger assembly 106. Once the needles are thrown through the tissue and the=sutures are in place, the needle and suture withdraw into the device using a retrieval mechanism 108. Alternatively, variations of the device include passive retrieval assemblies that decouple the suture from the needle assembly once the suture is properly placed through tissue. The needle assembly can then be retracted back into the main body leaving only the suture affixed within one or more retrieval channels 112. Other variations of the device include passive retrieval assemblies where the thrown needle picks up the stored suture and retrieves the suture to properly place it through tissue. In addition, the handle portion 104 can also include ports or couplings for fluid, suction/vacuum, drug delivery, or similar items that require coupling to the device 102.

As discussed below, the main body 102 of the suturing driving assembly 100 includes any number of constraining channels 110 and retrieval channels 112 that open at a tissue engaging surface 114.

The suture driving assembly 100 drives one or more pre-shaped needles (not shown in FIG. 1) through tissue in a manner that allows the pre-shaped needle to revert to its natural state or shape prior to entering tissue. This aspect allows the needle to be first maintained in a pre-deployment shape within a constraining channel 110 and yet deployed from the assembly 100 in the natural state. Such deployment permits the needle (and any attached suture) to pass through tissue in a predetermined path as defined by the natural shape without requiring deformation of the needle.

The suture driving assembly 100 of the present variation can also include an expandable device 200. In the illustrated example, the expandable device 200 comprises an expandable member (in this variation the expandable member is a balloon but the expandable member can be an expandable spline basket, an expandable funnel, a stent-like structure, etc.) 202 affixed to a shaft 204. Additional variations include an expandable member 202 comprising a mechanical basket, a fan shaped element, or any number of expandable structures commonly used in medical applications to secure tissue to a particular surface.

The shaft 204 can be axially moveable relative to the main body 102 so that tissue can be captured between the expandable member (or balloon) 202 of the expandable device 200 and the tissue engaging surface 114. Such movement can occur via a surgeon withdrawing the proximal end of the expandable device 200. Moreover, the variations of the assembly 100 can include various mechanism to lock the position of the expandable portion 202 with respect to the tissue engaging surface 114 so that a surgeon does not need to maintain constant tensile force on the expandable device 200. In addition, the expandable device 200 can include a guide wire lumen to assist in placing the expandable member 202.

As noted above, the tissue engaging surface 114 can also include any number of means to assist with securing tissue 2 against the tissue engaging surface 114 of the main body 102. For example, the tissue engaging surface 114 can include a number of vacuum or suction ports to affix tissue to the surface 144. Moreover, the tissue engaging surface 114 can be rough, channeled, or have other relief contours to move fluid or other substances away from the surface.

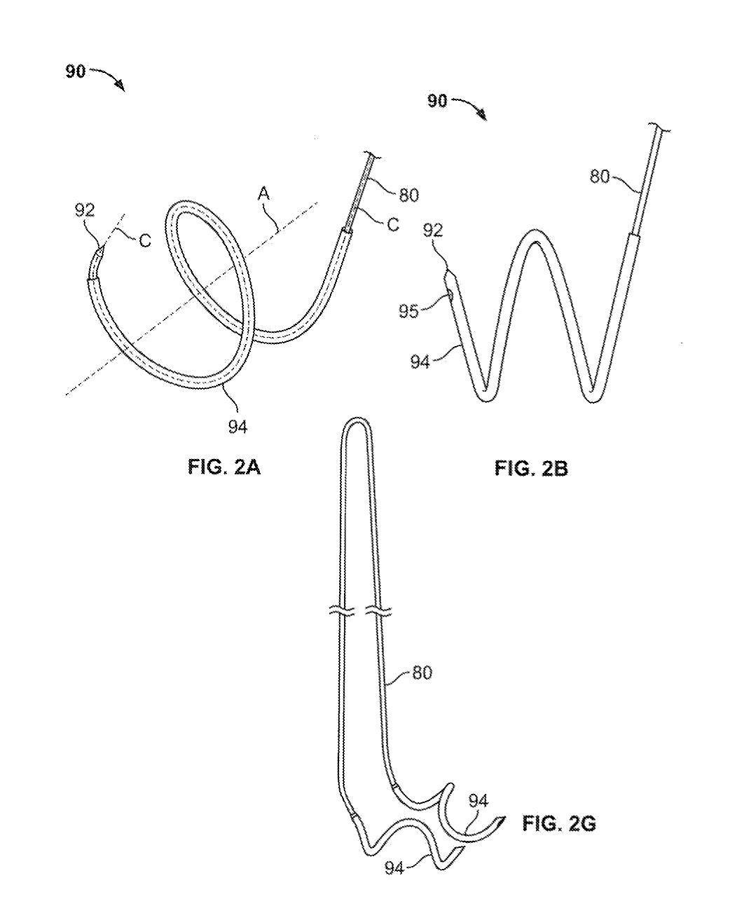

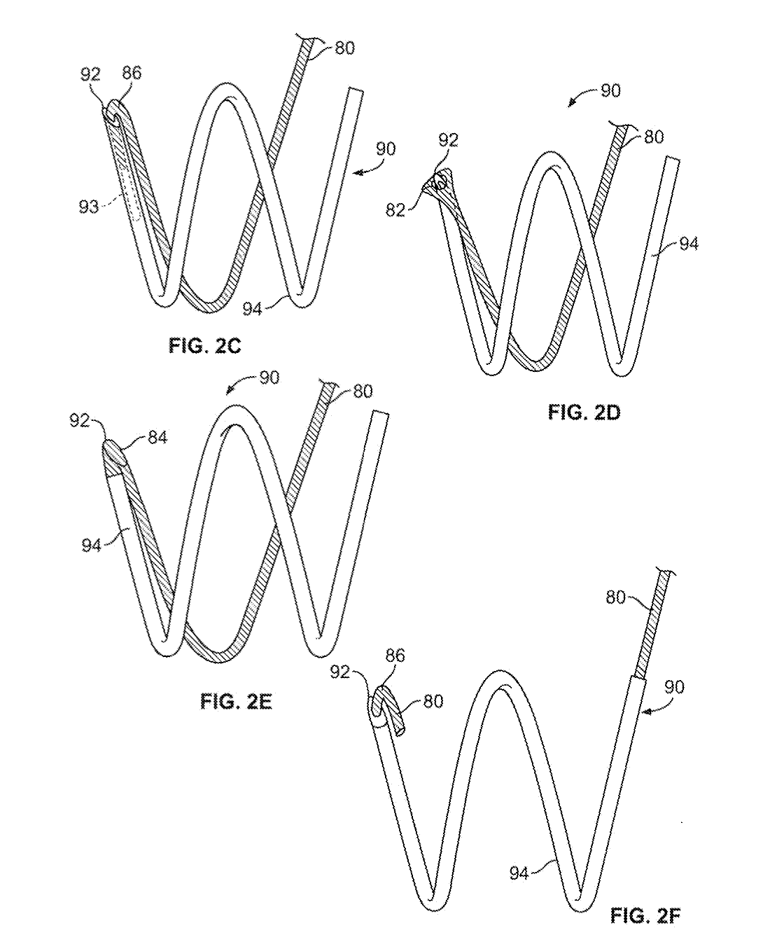

FIGS. 2A and 2B show isometric and side views of one variation of a needle or needle assembly 90. As will be discussed below, the suture driving assembly 100 can include any number of needles or needle pairs depending upon the desired application.

The needle assembly 90 typically comprises a tissue piercing end 92 distal to an elongate shaped section 94. The guide or shaped segment located within the main body allows the elongate shaped section 94 to revert to its natural shape prior to entering tissue as the piercing end 92 and shaped section 94 exits from the main body. The needle assembly 90 also includes a suture 80 coupled thereto. The shaped section 94 of the needles of the present devices includes a curvilinear shape. This shape can be planar (such as a curved needle), or can be three dimensional (as shown by the helix curvilinear shape that wraps about axis A). As noted above, the shaped section 94 of the needle assembly 90 comprises a center line C. In certain variations of the device, the angular bend of the shaped section 94 matches a centerline of the guide or shaped segment located to permit the shaped section 94 to revert to the natural curvilinear shape from a constrained state.

In addition, the shaped section 94 of the needle assembly 90 is elastically deformable into a pre-deployment shape when in the constrained state. Upon release, the shaped section 94 assumes its pre-set curvilinear shape. The needle assembly 90 can also include various features to aid in removal of the needle or suture from the body. For in certain variations of the device, the needle assembly 90 can include a notch, groove, or shoulder adjacent to the tissue piercing tip 92 where the notch 95 increases the ability of a retrieval assembly to withdraw the needle and/or suture. As illustrated, a suture 80 can be “back-fed” into the shaped portion 94. The suture 80 can be glued, crimped, or otherwise affixed to the shaped portion 94.

In some variations, it is desirable to have a needle assembly that does not contain any notch or openings that create areas of increases stress and create a risk of fracture areas. Accordingly, FIGS. 2C to 2E illustrate side views of needle assemblies having a “front-loaded” suture 80. As used herein, a front-loaded suture is one that can be removed from a front or tissue piercing end of the needle assembly. This configuration does not require the needle to be withdrawn through the retrieval channels in main body. Instead, once the needle assembly delivers the suture into the retrieval channel, because the suture is front loaded, it can be decoupled from the needle body at a front or tissue piercing portion. Such front-loaded sutures can be used with variations of the present suture loading device. FIG. 2C illustrates a suture 80 that is front-loaded into a lumen 93 of the needle assembly 90. The bend 86 of the suture 80 as it exits the lumen 93 holds the suture 80 against the shaped needle portion 94 causing the suture to reside within the lumen 93 adjacent to the tissue piercing end 92. Once the suture is advanced into a retrieving channel in the main body, retraction of the shaped section 94 causes the suture 80 to lift or decouple out of the lumen 93. FIG. 2D illustrates a suture 80 as having an opening or aperture 82 that can be hooked onto the tissue piercing end 92. FIG. 2E illustrates a cape 84 that is removably seated on the tissue piercing end 92. Clearly, any means of affixing the suture in a front loaded manner is within the scope of this disclosure. In such front-loaded configurations, rearward movement of the shaped portion 94 when the suture is held such as in the retrieval channel, causes the suture 80 to detach from the tissue piercing end 92. The sutures of the front-loaded needle assembly variations extend along an exterior surface of the shaped portion of the needle. As a result, the shaped portion 94 can be retracted or withdrawn while the suture 80 can be advanced into the device and ultimately secured or otherwise tied to accomplish closure of the opening in tissue.

FIG. 2F illustrates another front-loaded suture design where the suture extends through the shaped section 94 of the needle assembly 90. The suture 80 can have an optional bend 86 to secure the suture 80 against the needle assembly 90 during advancement. Alternatively, the needle assembly can have an increased frictional surface in the needle lumen to maintain the suture within the needle lumen when advancing the needle assembly through tissue. The remainder of the suture 80 extends through the shaped section 94 and out of a proximal end of the shaped section 94. Once the tissue piercing end 92 enters a retrieval channel, a suture retrieval device can secure the suture so that the needle assembly 90 can be retracted into a constraining channel.

Although the needles are shown having a helical shape, any number of curvilinear shapes are within the scope of the disclosure. For example, the shapes may be in a single plane or extend to form a 3-dimensional shape. In addition, the curvilinear shapes may have a plurality of curves, a single curve, and/or can be a partial circular shape.

The tissue piercing end and/or curved shaped section 94 can be comprised of a spring steel or other alloy that is set into shape. Alternatively, memory alloys can be employed. Such alloys include superelastic nickel-titanium (NiTi), copper-aluminum-nickel (CuAlNi), copper-zinc-aluminum (CuZnAl), or other shape memory alloys that are well known in the art.

FIG. 2G shows a variation of a needle assembly having two shaped sections 94 affixed to a single suture 80. This particular configuration is useful to produce a “mattress” stitch pattern. Accordingly, the associated suture driving assembly 100 will include multiple constraining channels as well as guide segments.

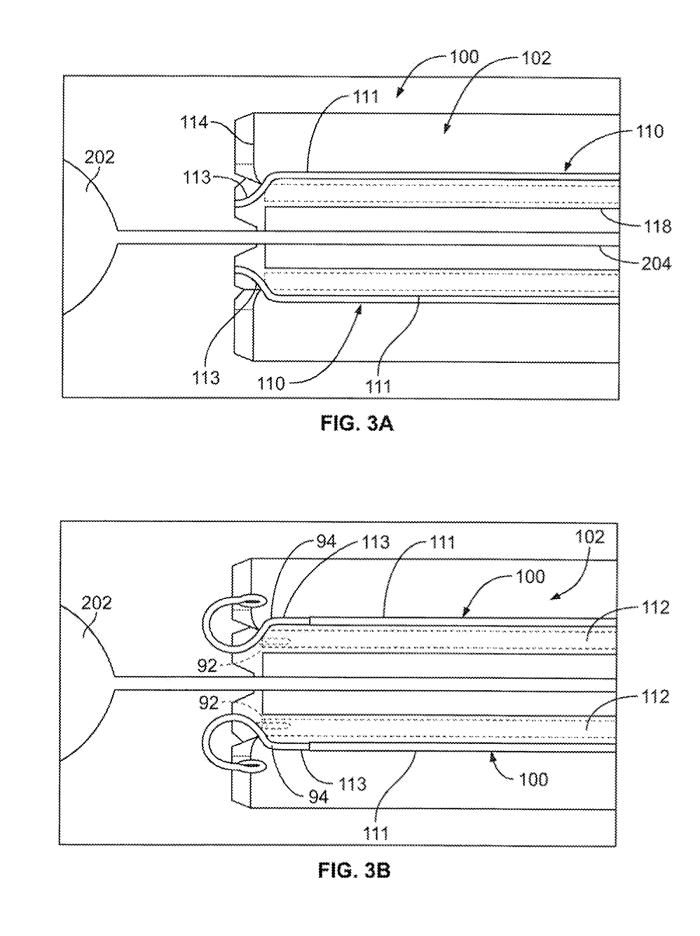

FIG. 3A illustrates a partial cross sectional view of a distal portion of a suture driving assembly 100. As shown, in this variation, the main body 102 includes a main lumen 118 through which the expanding member 202 and shaft 204 can advance. The main body 102 also includes any number of constraining channels 110 that terminate at a tissue engaging surface 114 of the main body 102. The constraining channel 110 can extend fully or partially through the main body 102. A needle assembly, and in certain variations a suture, can be loaded within the constraining channel 110 (though for the sake of illustration the suture and needle assembly are omitted from this figure). The constraining channel 110 comprises at least a restraining portion 111 having a profile to maintain the elongate shaped section into the strained state. In the illustrated variation, the guide portion is linear. However, variations of the device include restraining portions 111 having a variety of shapes.

The restraining portion 111 of the constraining channel 110 transitions into a guide segment 113 that is adjacent to the tissue engaging surface 114. The guide segment comprises a shape or a profile that matches the curvilinear shape of the shaped section of a needle. As a result, as the needle exits the main body, the needle passes through a guide segment having a curvilinear shape that allows the shaped section of the needle assembly to revert to its unconstrained curvilinear shape. Therefore, the needle assembly passes through tissue in its unconstrained state to the retrieval channels as illustrated below. FIG. 3B illustrates the shaped section 94 of a needle assembly as it passes through the guide segment 113 of a constraining channel 110. Once the needle assembly 90 is advanced a sufficient distance, the tissue piercing end 92 enters the retrieval channel 112. As discussed below, the needle assembly 90 can be advanced through the retrieval channel to a retrieval device (not shown) that pulls the needle assembly and suture through the main body. Alternatively, the needle assembly 90 can be retracted back into the constraining channel 110 leaving the suture secured within the retrieval channel 112. Doing so prevents the need of having to retrieve the entire through the main body. Instead, the needle assembly 90 can be withdrawn or retracted into the main body so that only the suture(s) must be retrieved through the main body.

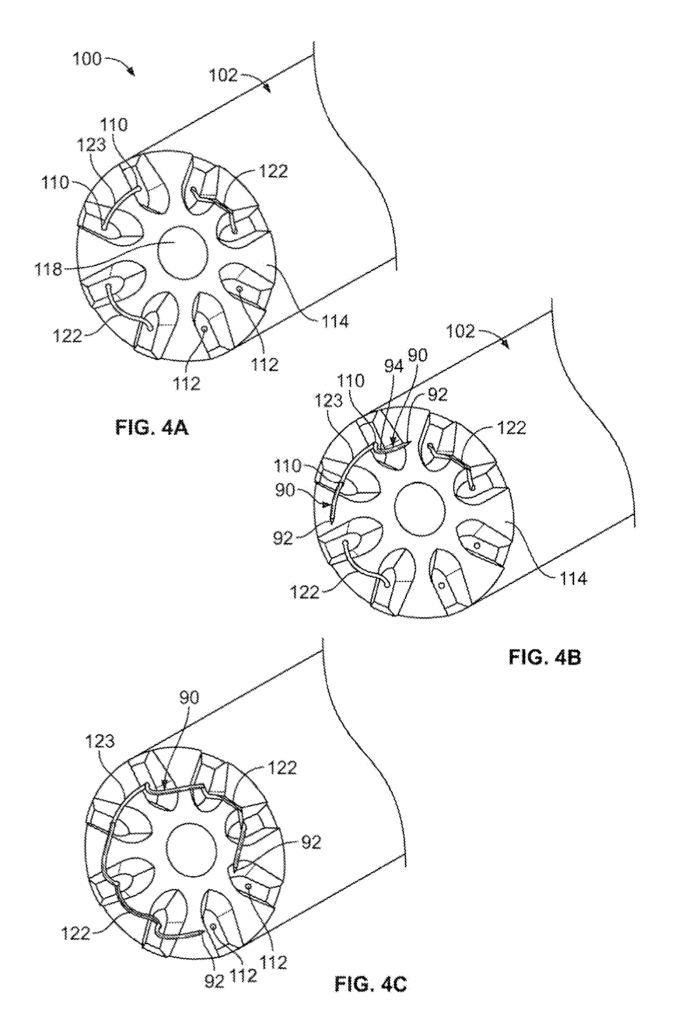

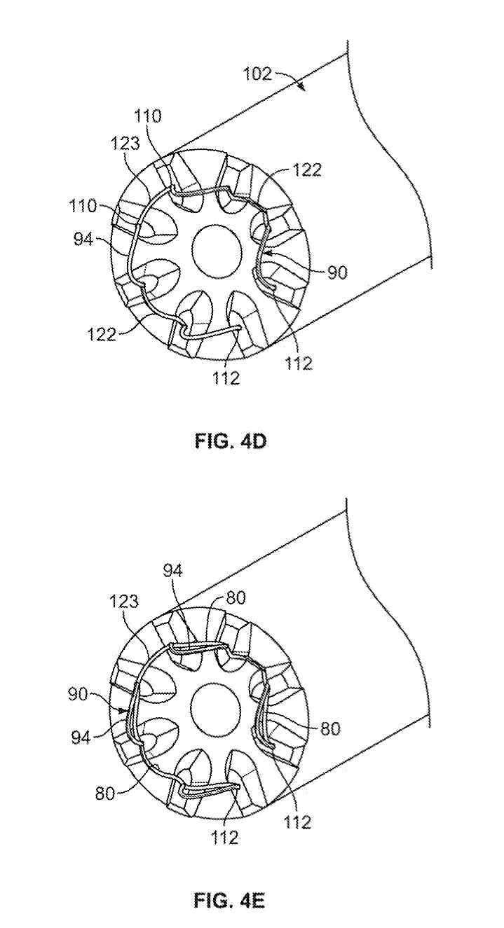

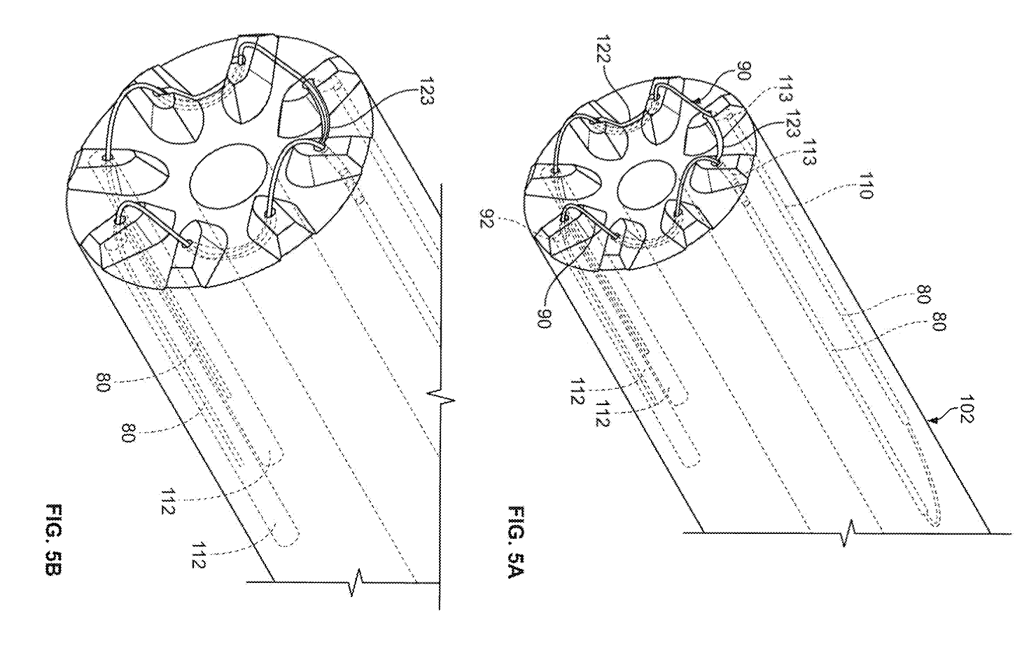

FIG. 4A illustrates a perspective view of a working end of a main body 102 of a suture driving assembly 100. As shown, the main body 102 can include a tissue engaging surface 114 having a number of constraining channels 110 and retrieval channels 112 with respective openings in the tissue engaging surface 114. The number and spacing of the constraining and retrieval channels 110 and 112 will vary depending upon the type of stitch or suture pattern sought. In addition, the sizing of the openings of the constraining and retrieval channels 110 and 112 can also vary.

As discussed above, the tissue engaging surface 114 can be flat, funneled, concave (as shown), or otherwise shaped to ensure proper tissue contact for insertion of a suture. Moreover, the tissue engaging surface 114 can include protrusions 116, channels, or other features to allow fluid to move away from the tissue engaging surface or to better compress the area of tissue in which a needle assembly is to be placed. The main body 102 can also include features such as channels 122 to direct the needle assembly through tissue. Furthermore, the main body 102 can include a main lumen 118 for delivery of an expanding device (not shown) as well as other medical tools/devices. Such a lumen 118 is required in those variations of the device configured for performing procedures within an organ or providing an access path within the organ. FIG. 4A also shows a relief opening 123 between the openings of adjacent constraining channels 110. A relief is typically used in variations of the device having a single suture joined to two needle assemblies 90. The relief opening 123 allows a suture that is joined by two needles to exit the main body when the suture is held within the main body 102. As shown below, variations of the main body 102 can include one or more suture relief openings on a side as well.

FIG. 4B illustrates the main body of FIG. 4A after a needle assembly 90 having two shaped sections 94, is advanced from constraining channels 110 of the main body 102. As discussed herein, the constraining channels 110 can contain a segment adjacent to the opening in the tissue engaging surface 114 that allows the shaped section 94 of the needle assembly 90 to revert to its unconstrained state. This allows the needle assembly to pass through tissue in a manner that is pre-determined by the curvilinear shape of the needle assembly 90. In the illustrated example, the needle assembly 90 is similar to that shown in FIG. 2G. As a result, in the illustrated example, as the shaped sections 94 revert to their unconstrained shape or profile, they orient in a helical curvilinear shape. In order to further direct the needle assembly 90 towards a respective retrieval channel 112, the tissue piercing end 92 of the shaped section 94 enters a guide path or guide channel 122. As shown in FIG. 4C, the guide path 122 deflects the shaped section 94 towards the retrieval channel 114 such that continued advancement of the needle assembly 90. FIG. 4D illustrates the device of FIG. 4C where the tissue piercing ends of the needle assembly 90 are advanced into the retrieval channels 112 in the main body 102.

FIGS. 4A to 4D illustrate one example of a suture advancing device where the suture follows the shaped section 94 of the needle assembly 90. However, as discussed above, variations of the device can include sutures that are front loaded within the needle assembly. FIG. 4E illustrates such an example after the needle assembly 90 is received within the retrieval channel 114 a suture 80 extending along an exterior of the shaped section 94 of the needle assembly 90.

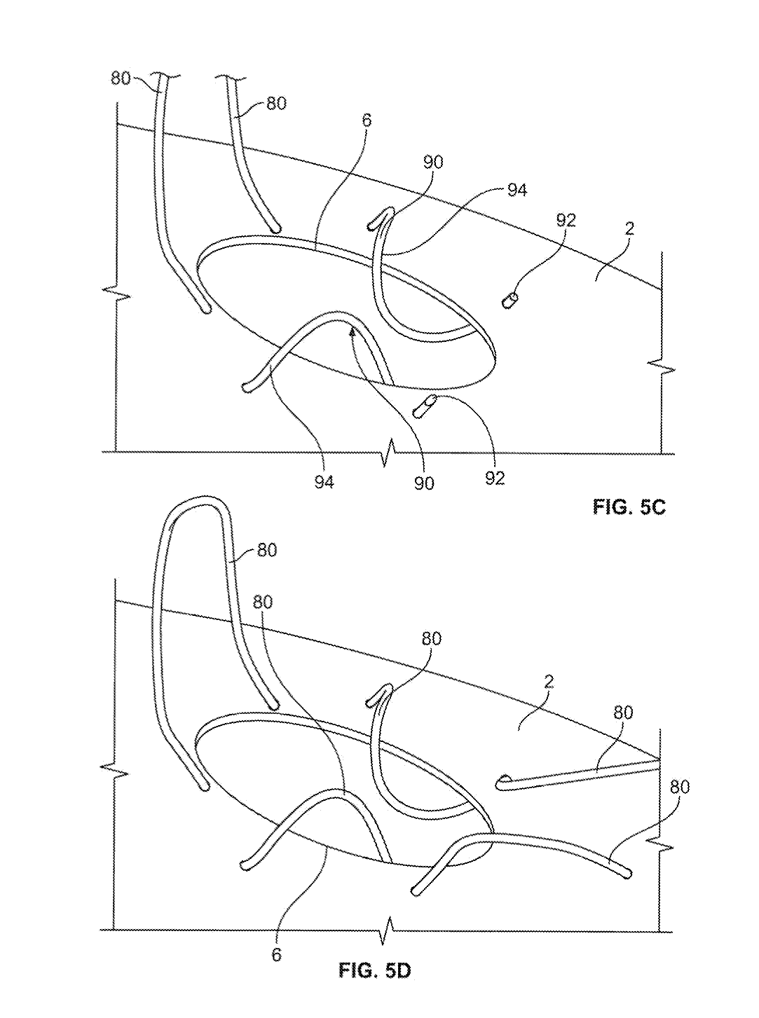

FIGS. 5A and 5B illustrate partially cross-sectional perspective view of a main body 102 having a needle assembly 90 with two shaped sections that is advanced between guide segments 113 of a constraining channels 110 and retrieval channels 112. As shown, the shaped portions of the needle assembly 90 are coupled by a single suture 80. As a result, as the needle assembly 90 leaves the constraining channels 110, the mid section of the suture exits the main body 102 via a suture relief opening 123. The ends of the suture 80 are located within the retrieval channels 112 where any number of mechanisms can be used to withdraw the suture ends.

FIGS. 5C and 5D illustrate respectively, the path of a variation of a needle assembly 90 and suture 80 when advanced in the manner shown in FIGS. 5A and 5B for closing an opening 6 in tissue 2. To clarify the path of the suture and needle assembly, the suture driving assembly is not shown in FIGS. 5C and 5D. As illustrated, the shaped sections 94 of the needle assembly 90 passes through tissue 2 in its unconstrained shape. In the variation shown in FIG. 5C, the needle assembly 90 is coupled to a single suture 80 and pulls the suture 80 through the tissue. FIG. 5D illustrates the state of the tissue 2 after the needle assembly 90 passes from the tissue 2 leaving only the suture 80 remaining in tissue. The resulting laced suture 80 passes through tissue about an opening 6 in the tissue but prior to tightening of the suture 80. Once the suture is “thrown” about the opening 6, the physician can secure the suture to close the opening 6. This particular suture pattern, when tightened, results in a purse string stitch. Clearly, devices within the scope of this disclosure can include any number of tissue receiving openings.

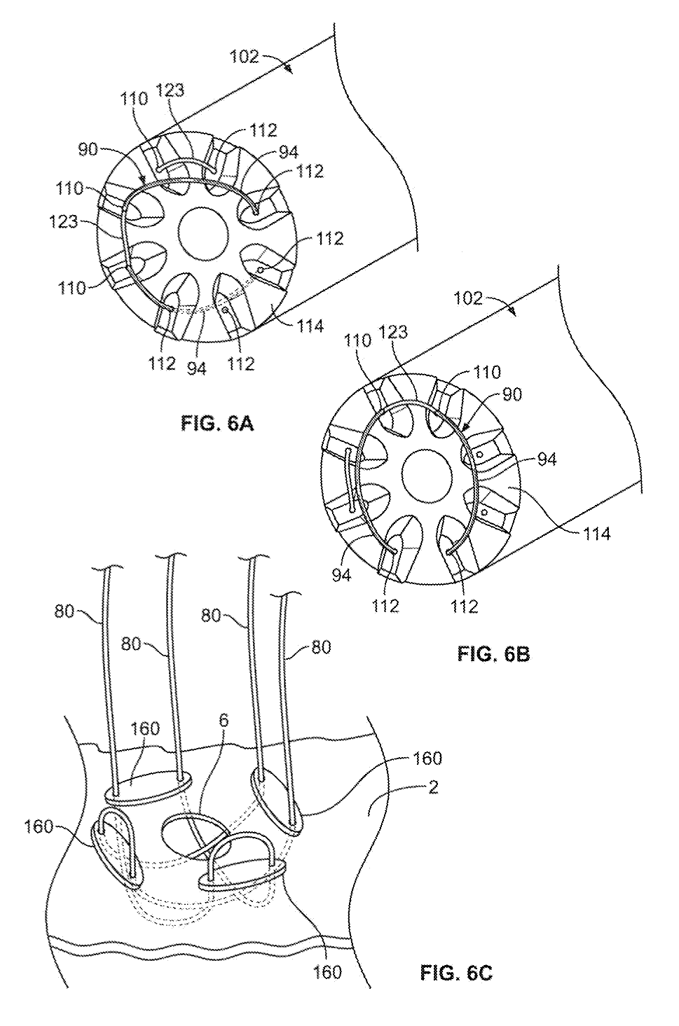

As discussed herein, the configuration of constraining and retrieval channels can be configured in any number of different variations to produce suture patterns as desired. For example, FIGS. 6A to 6C illustrate another such example. Clearly, any number of variations is within the scope of this invention with the illustrated variations depicting some possible variations.

FIGS. 6A and 6B provide another variation such of a main body 102 according to the present disclosure. In this variation, the main body 102 includes two adjacent pairs of constraining channels 110 and two adjacent pairs of retrieval channels 112 having openings in the tissue engaging surface 114. Though each of the openings of the individual pair of constraining channels 110 are joined by a suture relief passage 123, additional variations might not have these passages 123. As noted herein, such passages 123 are required when using a single suture between adjacent shaped sections of a needle assembly. FIG. 6A illustrates a first needle assembly 90 passing between a pair of constraining channels 112 and a pair of retrieval channels 112. FIG. 6B illustrates a second needle assembly 90 passing between the adjacent pair of constraining channels 112 and retrieval channels 112. Although the figures depict the needle assemblies being thrown sequentially, certain variations of the device allow for throwing the first and second needle assemblies at the same time.

FIG. 6C illustrates the sutures 80 depicted in FIGS. 6A and 6B once thrown and when the suture driving apparatus is removed from the tissue 2. As shown, the configuration of FIGS. 6A and 6B produce two perpendicular placed horizontal mattress stitches (one the sutures are properly secured). The illustrated variation also depicts the use of supports or surgical pledgets 160 that can be delivered on the tissue engaging surface of the main body and secured when the needle assembly passes through. Any variation depicted herein can include such pledgets (whether such pledgets are individually spaced about the opening or fully encircle the tissue opening).

Surgical pledgets can comprise biocompatible material (including polyamide, polyethylene, polypropylene, polyethylene terephthalate, polyurethane, olytetrafluoroethylene, various bioresorbable polymers and/or small pieces of autologous tissue. These pledgets are typically used in with the surgical suture to distribute the force of the suture applied on the tissue over a larger area or to aid in steaming the leakage of bodily fluids such as blood that results from penetration of bodily tissue by a suture needle and suture.

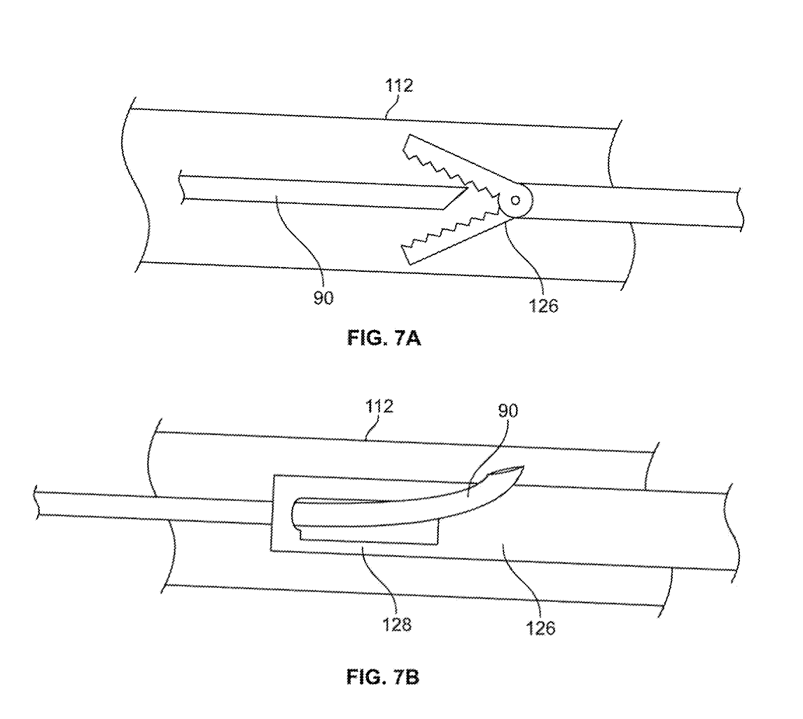

FIGS. 7A to 7B illustrate variations of suture retrieval device 126 that resides within the retrieval channel 112. As shown, as the tissue piercing end 92 of the needle assembly 90 enters the retrieval channel 112 variations of the assembly include a retrieval device 126. In FIG. 7A, the retrieval device 126 shows an example of a clamp or jaw type structure. FIG. 7B shows a retrieval device 126 including a window or slot 128 to capture the tissue piercing tip 92 (or a slot formed in the needle). However, the devices described herein are can include any retrieval device. For example, the retrieval device can comprise a cloth that is penetrated by the needle. The retrieval device 126 can be a finger-trap tubular type of device where tension applied to the device causes compression of the tube allowing for a pulling motion to secure the suture or needle for removal. The retrieval device can be a magnetic coupling device to also aid in removal of the needle or tissue piercing end. In addition, the retrieval devices disclosed in the references discussed in the background section can also be combined with the devices described herein.

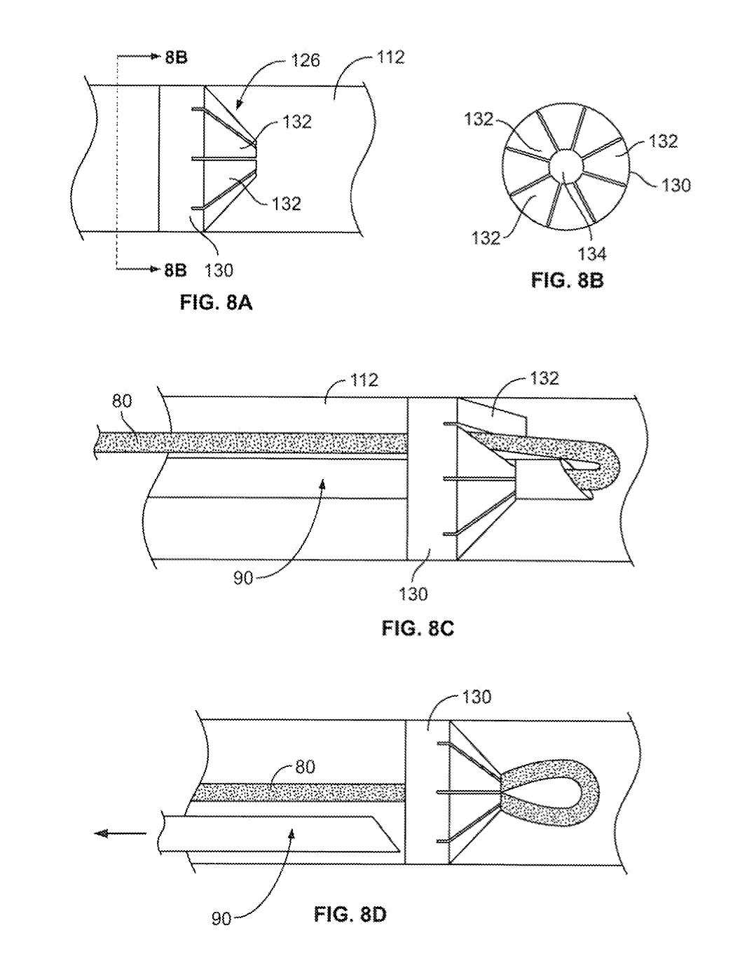

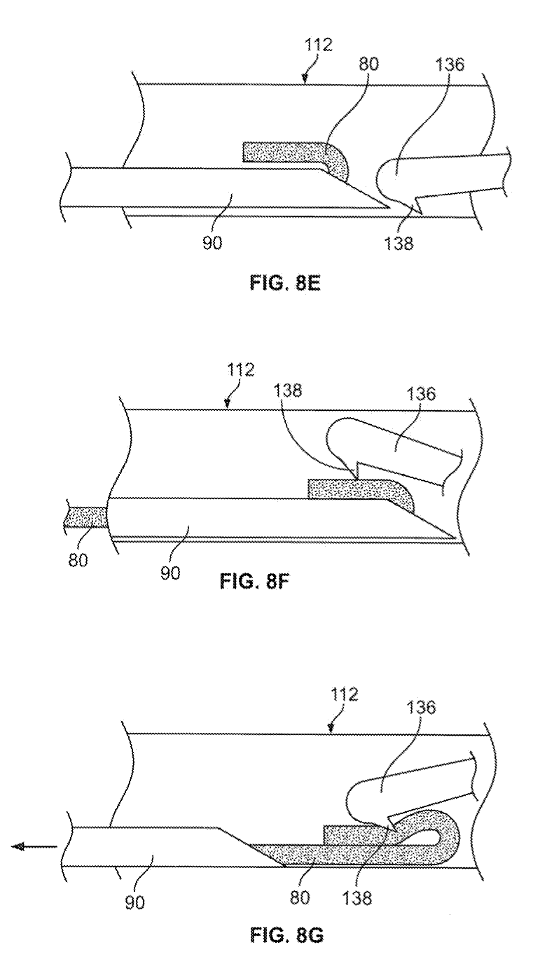

FIG. 8A illustrates another variation of a suture retrieving assembly 126 that comprises a pawl-type mechanism located in the retrieval channel 112. In the illustrated variation, the pawl-type mechanism comprises a slotted funnel 128 where the slots form sections 132 of the funnel that function as pawl-members. As shown by FIG. 8B, from the view taken along line 8B-8B from FIG. 8, the funnel sections 132 form an opening 134 that restricts a diameter of the retrieval channel 112. As shown in FIG. 8C, when a front-loaded suture 80 is loaded into or on a needle assembly 90, the arms 132 of the funnel 130 expand to allow passage of the suture 80 and needle assembly 90 through the funnel 130. However, the funnel members 132 function as a pawl mechanism as they are biased to return to the natural state shown in FIG. 8A. Accordingly, as the needle 90 and suture 80 are retrieved, the arm member (or members) 132 frictionally engage the suture 132 and can compress or bite into the suture. The relatively rigid nature of the needle assembly prevents the funnel members 132 from preventing rearward movement. Accordingly, withdrawing the suture 80 and needle assembly 90 dislodges the front-loaded suture 80 and traps the suture within the funnel 130 as shown in FIG. 8D as the needle assembly 90 is withdrawn. In some variations, the funnel 130 is moveable within the retrieval channel so that the suture 80 can be withdrawn without moving the suture driving assembly. However, in cases where the funnel 130 is stationary, the entire suture driving assembly or the main body alone can be withdrawn to ready the suture for tying about a tissue opening.

Various additional pawl mechanisms are intended to be within the scope of this disclosure, for example, the paw mechanism can comprise a traditional pawl comprising of a hook or tooth located on an arm, where the pawl is biased to engage a suture as it enters the retrieval channel. For example, FIGS. 8E to 8G illustrate a pawl-type mechanism 136 that is biased within a retrieving channel 112 of a device to reduce a size of the channel 112. As illustrated in FIG. 8E, as the front-loaded suture 80 and needle assembly 80 enter the retrieval chamber, the pawl mechanism 136 interferes with the suture 80 and needle assembly 90. Because the pawl-mechanism 136 is spring biased, the suture 80 and needle assembly 90 deflect the pawl-mechanism 136. At this point, as shown in FIG. 8F one or more teeth or protrusions 138 on the pawl-mechanism 136 bite into the deformable suture 80. Once the pawl-mechanism 136 engages the suture 80, the needle assembly 90 can be withdrawn leaving the suture 80 secured within the retrieval channel 112. The pawl-mechanism 136 can then be withdrawn in the channel 112 or the entire device can be withdrawn to pull the secured suture. In addition, the surface of the retrieval channel 112 can have any number of protrusions, hooks, or other features to capture the suture or increase friction against a captured suture.

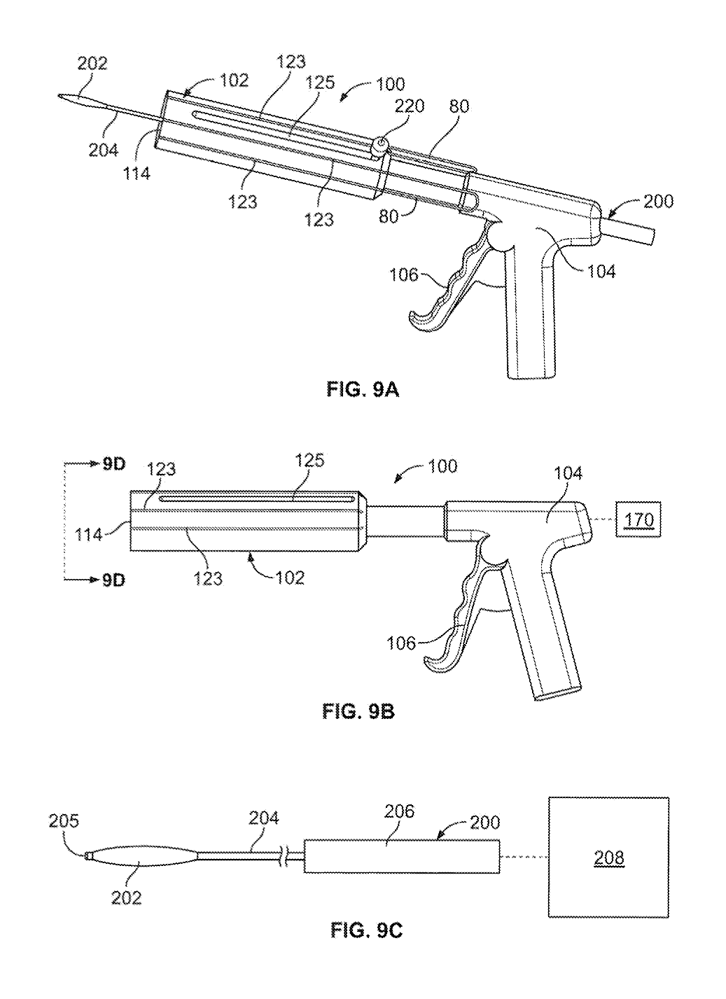

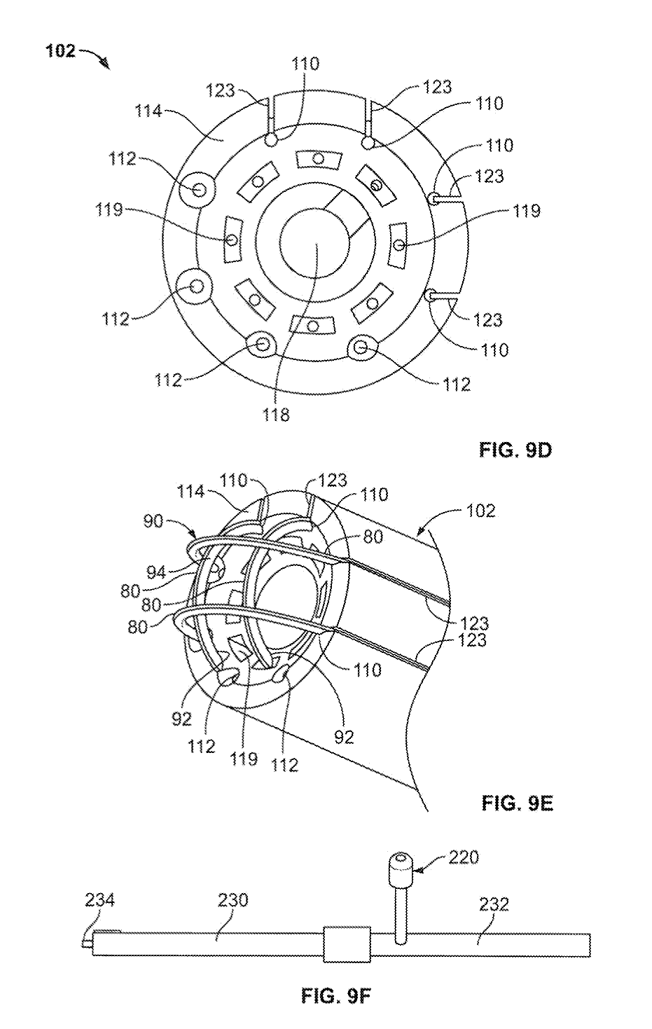

FIG. 9A shows another variation of a suture driving assembly 100 having a expandable device 200 extending through a main lumen. Again, the expandable device 202 can include a balloon 202 or other expandable member affixed to a shaft 204. The expandable device 202 can optionally be stationary within the main lumen or can be moveable relative to the main body 102. The present variation also optionally includes a pin or lever 220 that is moveable within the main body 102. As discussed below, the pin 220 allows advancement of an introducer or other device through the main body. Once the pin 220 is advanced to a desired location, the pin 220 can be removed from the main body 102 to de-couple the main body from the introducer/device located within the main body.

FIG. 9B illustrates the main body 102 of FIG. 9A. As shown, the main body 102 of the present illustration can include a number of suture channels 123 that extend along an exterior surface of the main body 102. As discussed above, such a feature allows a suture to ext the main body when both ends of the suture are joined to one or more needle assemblies that are advanced through tissue. In the illustrated variation and as shown in FIG. 9A, the main sutures 80 can optionally extend through a rear portion of the main body 80. Though not shown, the sutures could be wrapped about spools (not shown) or placed in protective tubing (not shown) rather than remain exposed. Alternatively, the suture channels 123 can extend only through a portion of the main body 102 where the section of suture leaving the suture channel 123 can then be affixed or seated in any portion of the suture driving assembly 100.

FIG. 9B also shows the suture driving assembly as only having a trigger 106. In such variations, the handle portion 104 and trigger 106 rely on a spring based mechanism so that once the trigger 106 is fully actuated, the spring based mechanism releases the trigger 106 and withdraws the needle assembly within the tissue engaging surface 114. As a result, manual retrieval of the needle assembly is not required.

FIG. 9B also illustrates the main body 102 as having a slot 125 to accommodate the pin 220 shown in FIG. 9A. The slot 125 can optionally extend through the proximal and/or distal ends of the main body to allow decoupling.

In those cases where the suture driving assembly 100 relies on a vacuum source 170 to assist in securing tissue against the tissue engaging surface 114, the handle portion 104 or main body 102 can be fluidly coupled to the vacuum source 170 by any conventional means. In addition, the suture driving assembly 100 can also be coupled to any additional fluid supplies to deliver medication, irrigation, or other fluids to the site of the tissue repair.

FIG. 9C shows the expandable device 200 of FIG. 9A. In the present variation, the expandable device 200 can include a shaft 204 having a guide wire lumen 205 extending therethrough. The shaft 204 can have sufficient column strength to allow a surgeon to manipulate a handle 206 at the end of the device 200 to advance the balloon 202 or other expandable member into the tissue being closed. Moreover, in those cases where the expandable device requires a fluid source 208, the handle 206 can include any number of fluid lumens and connectors to fluidly couple the fluid source 208 to the expandable device/balloon 202.

FIG. 9D illustrates the tissue engaging surface 114 as viewed along lines 9D-9D in FIG. 9B. For purposes of illustration, the handle portion and trigger are not shown. FIG. 9D shows the tissue engaging surface 114 of the main body 102 as being tapered or concave with a number of vacuum 119 ports located in the surface 114 and adjacent to both the main lumen 118 and the constraining and retrieval channels 110 and 112. As discussed above, some or all of the ports 119 can be coupled to other fluid delivery sources for irrigation or delivery of other substances.

FIG. 9D also shows two pairs of constraining channels 110 opening onto the tissue engaging surface 114. Each of the constraining channels 110 joins a suture channel 123 as shown in FIGS. 9A and 9B. Accordingly, as the suture advances through the main body, the mid section of the suture can travel outside of the main body 102 along the suture channels 123. The illustration also shows a number of retrieval channels 112 equal to the number of constraining channels 110. In this variation, the retrieval channels 112 have a tapered opening in the tissue engaging surface 114. The suture retrieval devices 126 discussed above can be located within the tapered opening or more distally in the channel 112.

FIG. 9E illustrates the tissue engaging surface 114 of the main body 102 where two pair of needle assemblies 90 are partially deployed from the main body 102. In this variation, the needle assembly pairs 90 are located 90 degrees relative to one another. As shown, the curved section of the needle assemblies 94 comprise a curvilinear shape having a single curve. This variation of the needle assemblies 90 also includes a suture 80 that is front loaded into the needle assembly adjacent to a tissue piercing end 92. FIG. 9E illustrates the needle assemblies 90 just as the tissue piercing ends 92 and front loaded suture 80 are entering the retrieval channels 112. As discussed above, once the needle assemblies 90 enter the retrieval channels 112, a retrieval mechanism (not shown) secures the sutures 80 so that upon retraction of the needle assemblies 90, the sutures 80 remain within the tissue.

FIG. 8F illustrates one example of a device that can be advanced through a main body of a suture driving assembly. In this variation, the device comprises an introducer 230 located on a dilation device 232 having a dilation tip 234 extending from the introducer 230. Advancement of the dilation device 232 and/or introducer 230 can occur via manipulation of the dilation device 232 through a rear end of the suture driving assembly. However, the illustrated variation shows a pin/lever 220 that is irremovably coupled to the dilation device 232 so that the introducer 230 and dilation device 232 can be advanced via movement of the pin 220 as it extends from a slot in the main body as shown in FIG. 9A.

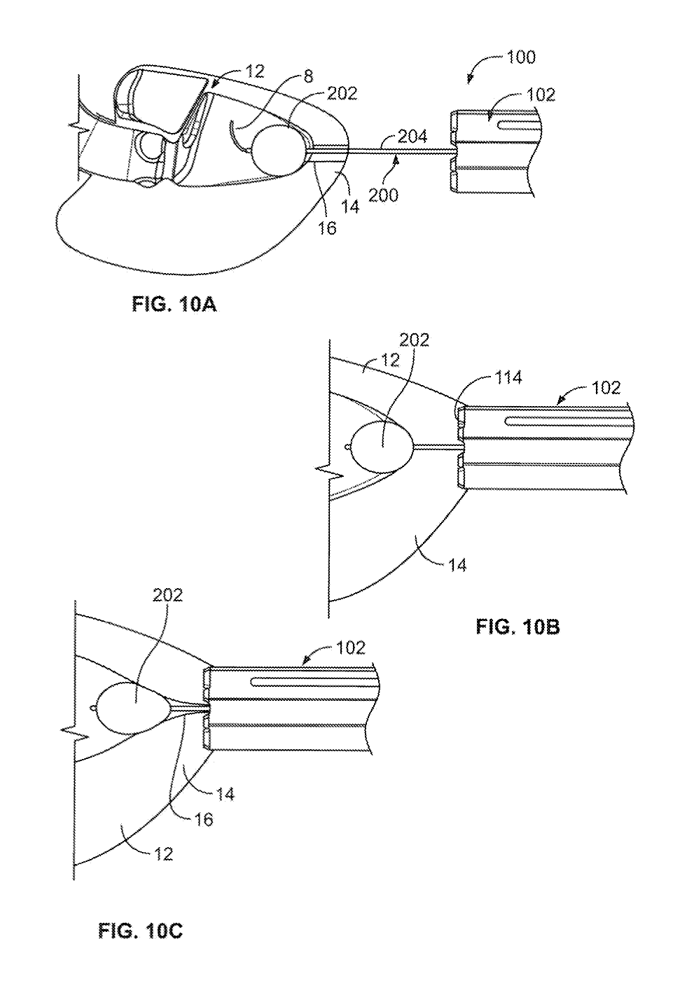

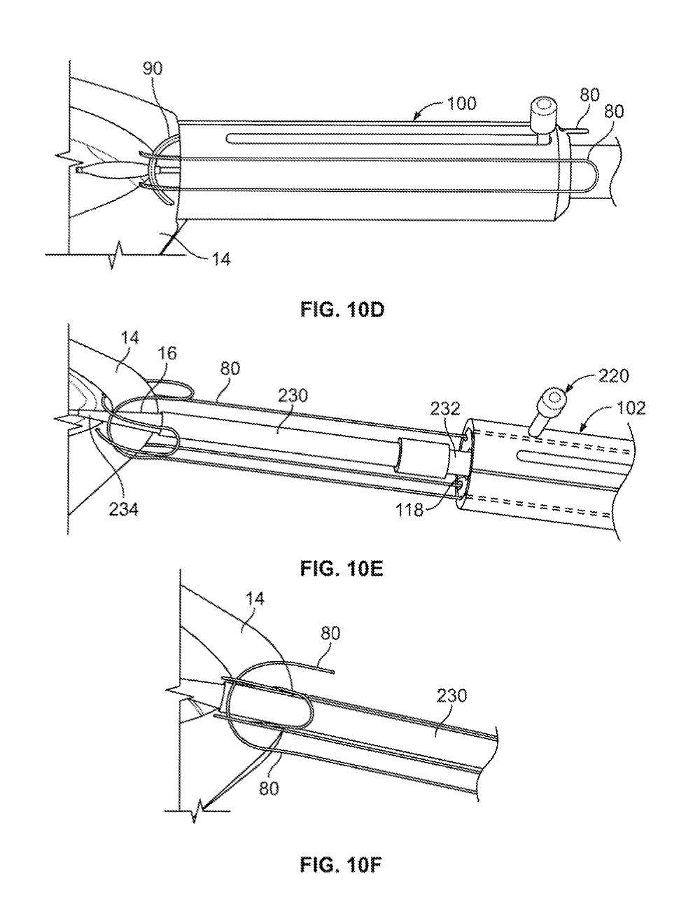

FIGS. 10A to 10E illustrate an example of a procedure for closing an opening in an organ. In this example, the organ comprises an apical portion 14 of a heart 12. However, it is within the scope of this disclosure that the suture driving assembly described above can be used in a variety of situations where closing of a puncture, tear, or opening in tissue is required and in any number of organs. When used in the illustrated apical approach, the suture driving assembly is useful for closing a puncture 16 in the heart's apex 14 after performing a trans-apical valve replacement or repair, when placing a ventricular assist device, or other procedure that would benefit from closing an opening in the heart. As illustrated below, the suture driving device can also be used to deliver additional devices to the tissue site.

FIG. 10A illustrates the heart 12 after a physician tracked a guidewire 8 into an apical portion 14 of the heart where an apical opening 16 allows access to the interior of the heart. Next, the surgeon tracks an expanding device 200 over the guidewire and into the opening at the apical portion 14. In this example the expanding device is a balloon catheter but as noted above, any type of expanding device can be used. Moreover, any traditional technique for tracking a guidewire and catheter can be employed to position the guidewire and expandable device into the heart 12.

Once the physician expands the balloon 202 within the heart 12, the physician can then advance a main body 102 of the suture driving assembly 100 a shaft 204 of the expanding device 200. At this time the surgeon can expand the balloon 202 to minimize dislodging of the assembly from the heart. As shown in FIG. 10B, once the main body 102 of the device 100 engages the apex 14 of the heart 12, the surgeon compresses the apex 14 between the balloon 202 and the tissue engaging surface 114 of the main body. As noted above, the main body can include any number of features to ensure good contact with the tissue. For example, the surgeon can draw suction through ports in the main body to ensure that the apical wall 14 secures to the main body 102.

The physician can optionally fully or partially deflate the balloon 202 (or reduce a diameter of other expandable structures if used). Once reduced, the expandable portion 202 or balloon can be retracted into the apical opening 202 as shown in FIG. 10C. This partial retraction of the balloon into the opening can further stabilize the device to the organ or tissue.

As illustrated in FIG. 10D, once the physician is satisfied with the placement of the device 100, the physician can actuate the device 100 to advance the needle assemblies 100 and throw the sutures 80 through the apical tissue. As noted herein, the needle assemblies can be fired simultaneously or sequentially. In some variations the balloon or expandable member remains inflated during advancement of the needles. In such cases, the balloon/expandable member can be fabricated so that the needle assembly will deflect away from the surface of the expandable member to prevent rupture or trapping of the needles. Moreover, the needle assemblies can fully penetrate the wall of tissue or can remain within the wall. The depth of the throw is typically a function of the type of tissue, the tissue engaging surface, and the design of the needle assembly. In cases such as an apical procedure, the needles may not need to fully penetrate the walls of the organ. For example, the needles in the present example do not reach the balloon but remain within the hearts apical tissue until they return to the retrieval channels.

After placement of the suture the main body 102 can be retracted to expose the suture ends outside the body cavity so that the surgeon can secure the sutures. The balloon can then be deflated and removed. The physician can then place an appropriate port/cannula through the apical puncture 16 and hold the port in place during the procedure by tightening of the stitch. After the procedure is complete, the port/cannula is removed and the purse string suture is drawn tight and secured with a knot or cinch to provide closure of the apical puncture.

FIG. 10E shows an alternative approach, in this variation the surgeon retracts the main body 102 and disengages any vacuum or suction being applied. Next, the surgeon advances a port or introducer sheath 230 the main lumen 118 of the main body 102 and ultimately advanced into the apical opening 16. As noted above, the introducer sheath 230 can be advanced using a pin 220 that slides through an opening in the main body 102. Alternatively, the introducer 230 can be advanced by manipulation of a proximal end of the dilator device 232. The dilator device 232 eases the transition through the apical opening 16 and ensures a seal between the apical opening 16 and introducer sheath 230. The expandable member/balloon can be removed prior to, or during insertion of the introducer sheath 230 within the heart. Alternatively, though not illustrated the balloon can remain in place until the introducer is secured.

As shown in FIG. 10F, once the physician places the introducer sheath 230, the surgeon withdraws the main body 102 to expose the sutures that were previously thrown in the tissue. Removal of the main body allows access to the sutures 80 so that the sutures can be tightened around the dilator 16 and/or introducer sheath 230. As a result, the sutures 230 are temporarily tensioned about the introducer sheath 230 to form a tissue seal around the introducer sheath 230. This temporary fixation allows the physician to create an access path into the heart for performing any appropriate procedures. Once the procedure is completed, the surgeon removes the introducer, and any other remaining devices as necessary while leaving the sutures 80 in place. Once all devices are removed from the opening, the physician secures the sutures 80 to close the apical opening. FIG. 6C provides an example of the suture pattern that will be left in the apical portion of the wall upon removal of the remaining devices.

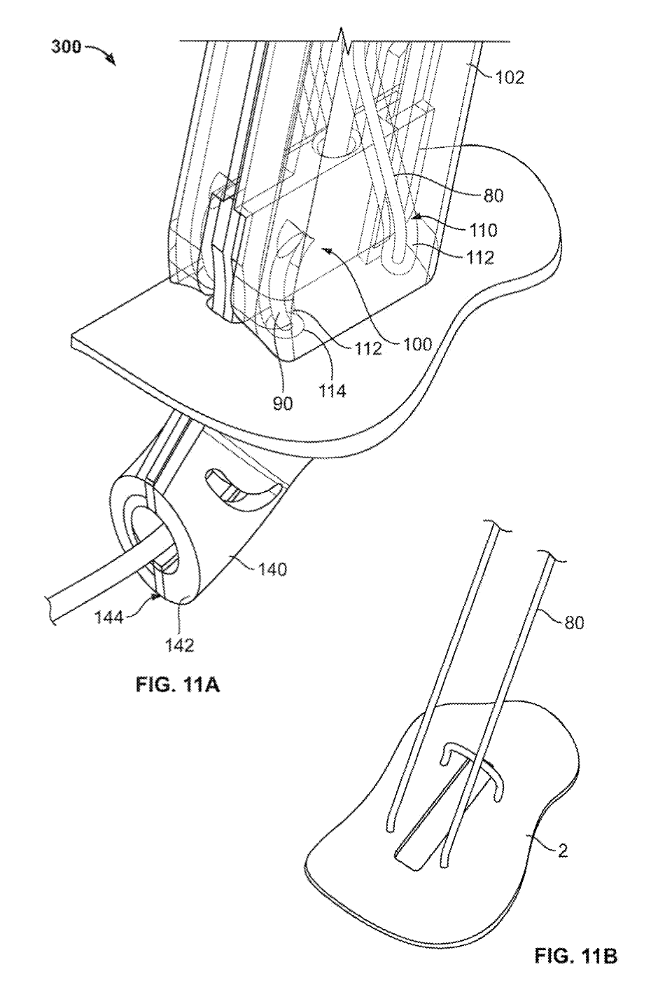

FIG. 11A shows an example of a distal end of a suture driving assembly 100. In this variation, the suture driving assembly 100 includes a main body 102. The suture driving assembly 100 drives a pre-shaped needle 90 through tissue in a manner that allows the pre-shaped needle to revert to its natural state or shape prior to entering tissue. This aspect allows the needle 90 to be first maintained in a pre-deployment shape within a constraining channel 110 and then deployed from the assembly 100 in the natural state. Such deployment permits the needle 90 (and any attached suture 80) to pass through tissue in a predetermined path as defined by the natural shape of the needle 90 without requiring permanent deformation of the needle.

The suture driving assembly 100 can optionally include a clamp assembly 140 to secure tissue 2 against the main body 102. In additional variations, the suture driving assembly 100 can include vacuum ports or other tissue securing structures (hooks, barbs, adhesive, etc.) in place of or in combination with a clamp assembly. Further detailed discussion of the clamp assembly 140 follows below. The clamp assembly 140 is useful for procedures where the procedure requires a high degree of certainty that tissue is retained against the main body 102. Securing the tissue in this manner assists in properly driving a needle 110 through tissue. In one example, such clamps can be used in vascular procedures. However, in alternate variations, a clamp assembly 140 can be omitted or replaced with either a non-expanding member or a shield type member that protects tissue from unintended advancement of the needle.

FIG. 11A also shows the suture driving assembly 100 with a constraining channel 110 extending along the main body 102. The constraining channel 110 shown is depicted as being within the main body 102. However, variations of the assembly 100 include a constraining channel 110 located along the outer wall of the main body 102, within a wall of the main body 102, exterior to the main body 102, or as a lumen in the wall of the main body 102. The constraining channel typically has a profile or shape that constrains a needle located therein to a pre-deployment shape. Constraining the needle in a pre-deployment shape allows for minimizing a profile of the suture driving assembly 100 (such as when the assembly must be advanced through small diameter access devices). The constraining channel 110 maintains a needle in a substantially straight profile when the needle is fully housed in the constraining channel 110. However, other profiles are within the scope of this disclosure.

The constraining channel 110 includes a guide segment 112 at a distal end. The guide segment 112 as discussed below allows for a needle constrained in the pre-deployment shape to recover to the natural shape. As the needle advances from the constraining channel 110, the portion of the needle within the guide segment 112 reverts to the path of least resistance in the guide segment 112 such that the needle leaves the guide segment in its natural shape. An example of this deployment feature is discussed in detail below.

The suture driving assembly also includes a suture retriever assembly (not shown) for withdrawing the suture 80 through the assembly 100 after the suture 80 passes through tissue. As discussed herein, some variations of the assembly withdraw the suture and needle from the assembly 100 while other variations withdraw the suture from the assembly while the needle retracts back within the constraining channel 110.

As illustrated in FIG. 11A, the guide segment 112 extends through and opens at the distal end of the main body 102. The assembly 100 also includes a needle receiving opening 114. Although the needle receiving opening 114 is shown as being within the main body 102 additional variations may be included. For example, a needle receiving opening 114 can comprise a separate channel, tube, or lumen that extends through or along the main body 102.

FIG. 11B shows the suture 80 placed in a horizontal mattress stitch pattern in the tissue 2 when the suture driving assembly includes a pair of needles tethered by a continuous suture loop. In this variation, two needles were attached to a single length of suture to drive the ends of the suture as shown. The middle portion of the suture then engages the tissue.

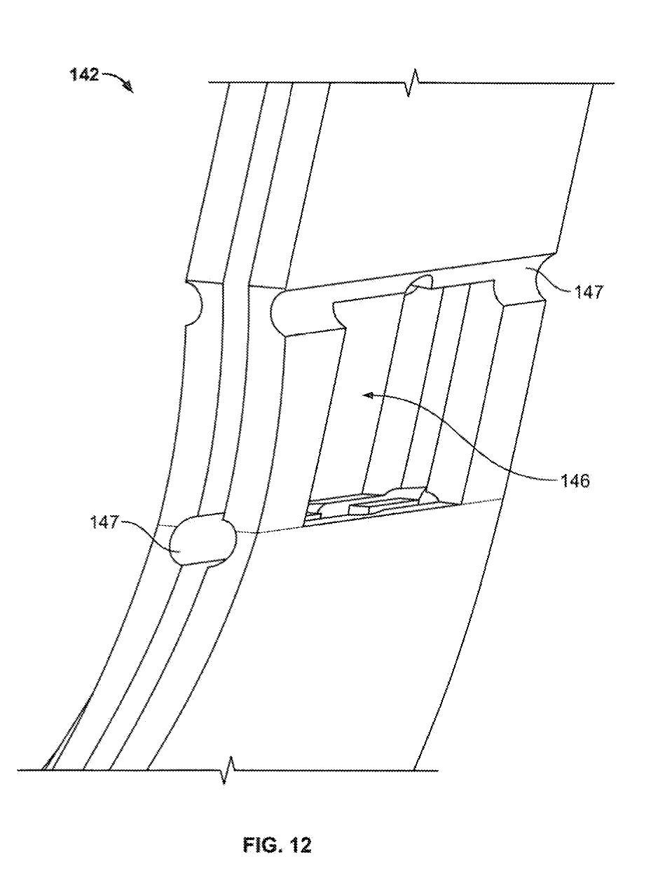

FIG. 12 illustrates one variation of an expandable clamp body for use with a suture driving assembly. The clamp body 142 can have weakened or thin sections 147 in the wall to create a living hinge that is integral to the clamp body 142. The clamp assembly 140 includes a clamp body 142 at a distal end of a clamp plate 144. The distal end of the clamp body 142 is secured to the clamp plate 144, while the proximal end of the clamp body 142 is fixed to the main body 102. To flex the clamp body 142 and expand it in a lateral direction, the clamp plate 144 is retracted axially. Furthermore, the clamp assembly 140 can include additional features such as a groove, recess, or window 146 to accommodate passage of the needle assembly 90. In some variations, the clamp assembly 140 also includes a lumen 148 extending therethrough so that the suture driving assembly 100 can be advanced over a guidewire. However, the lumen can be used for any number of purposes.

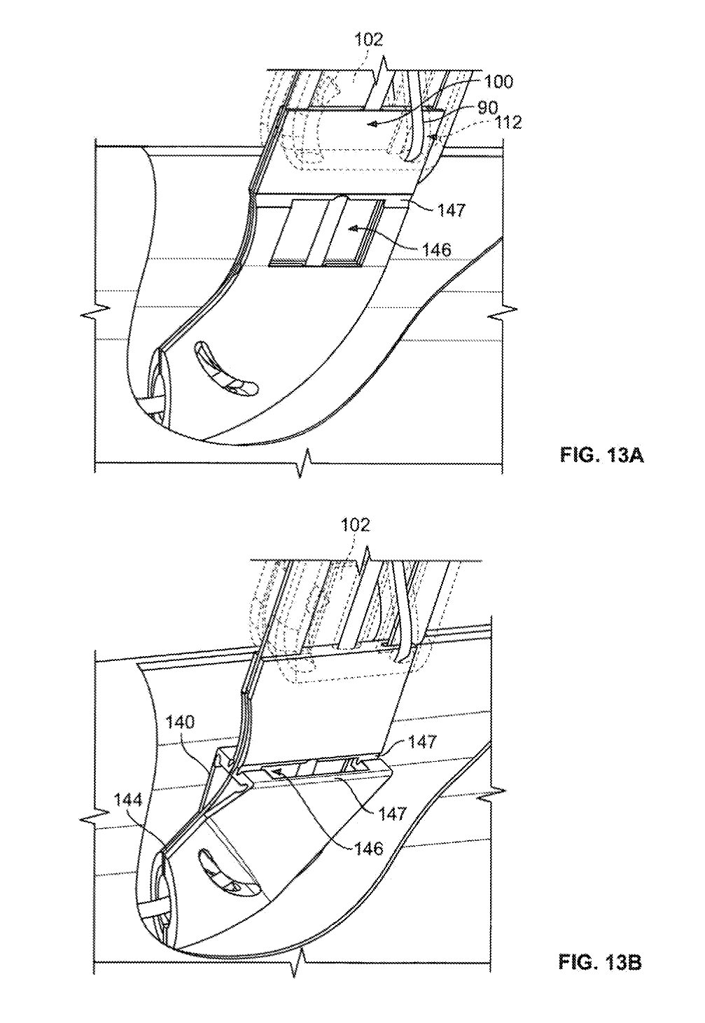

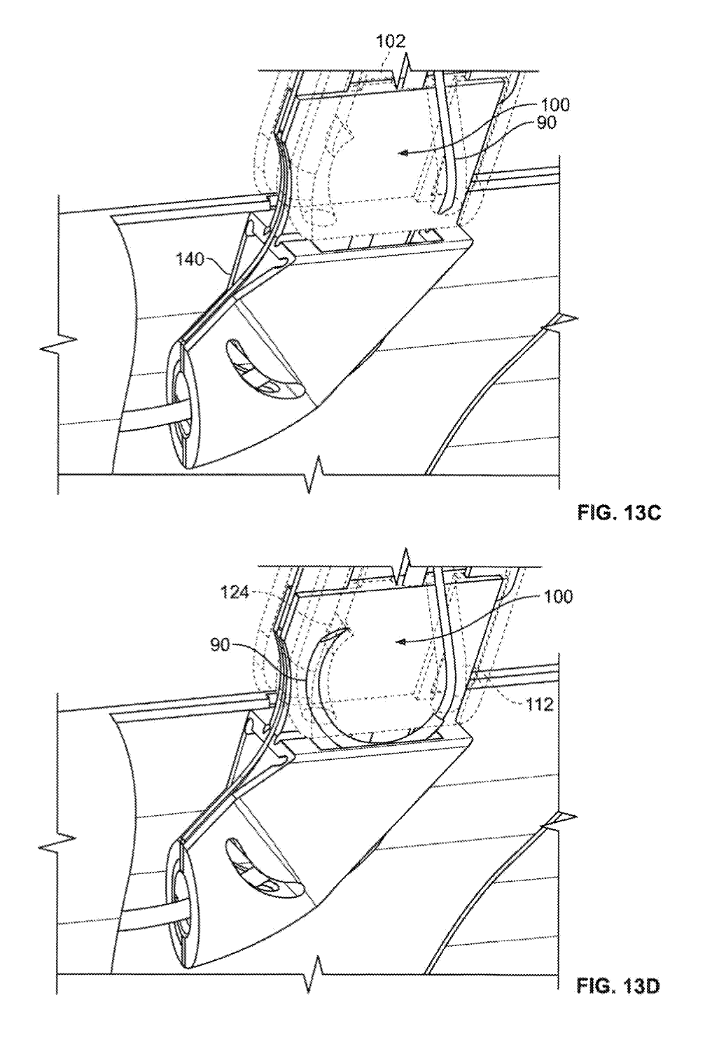

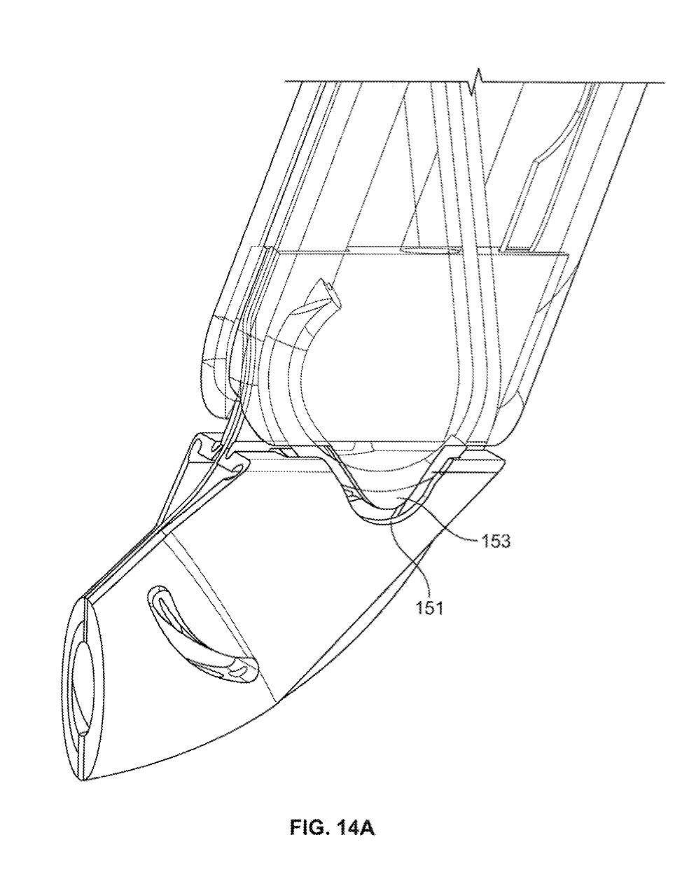

FIGS. 13A to 13D show an example of a suture driving assembly 100 advancing a needle assembly 90. In FIG. 13A, the needle assembly 90 is shown within the constraining channel 100 and has a suture (not shown) extending from the proximal end of the needle assembly 90. As noted above, the guide segment 112 of the constraining channel 110 has a different shape than the remainder of the constraining tube 110 that permits the needle assembly 90 or curved portion 94 to revert to the natural orientation of the needle assembly 90 once the needle assembly 90 moves out of the constraining channel 110.

FIG. 13B shows the needle assembly 90 still within the constraining channel 100. However, the clamp plate 144 is now retracted axially, creating a compressive force to flex the clamp body 142 and expand it in a lateral direction. FIG. 13C shows the entire clamp assembly 140 has been retracted toward the main body 102, securing the tissue.

FIG. 13D shows the needle assembly 90 continuing to exit from the guide segment 112 in the natural unconstrained pre-set shape. Since the needle assembly 90 moves through its natural curvilinear shape, the path of the suture through tissue will follow the curvilinear shape. The needle assembly 90 reverts to its natural shape as it continues to move through its natural shape. The needle assembly 90 then enters the needle retrieving opening 124 within the main body. The tissue piercing end 92 of the needle assembly 90 enters a retrieval device (not shown). The retrieval device can include any rigid type clamp or jaw structure that is disclosed in the references discussed in the background section. The retrieval device can be a finger-trap tubular type of device where tension applied to the device causes compression of the tube allowing for a pulling motion to secure the suture or needle for removal. The retrieval device can be a magnetic coupling device to also aid in removal of the needle or tissue piercing end.

As noted above, variations of the devices according to the present disclosure can include constraining channels can extend in a linear fashion, as well as a non-linear manner about the device. For example, the constraining channels could be attached to a pivoting member that swings away from the main body to enhance the suture entry location. Any such configuration can be employed so long as the restraining channel maintains the needle assembly 90 in a pre-deployment shape. The restraining channels may also slideably reside within or be attached to a piercing rod that is movable in relation to or extends beyond the distal end of the main body. The piercing rod may be advanced prior to advancing the needle assembly to pierce the tissue and place the distal end of the restraining channel below the surface of the tissue. The restraining channel could have a sharpened distal end and act as the piercing rod.

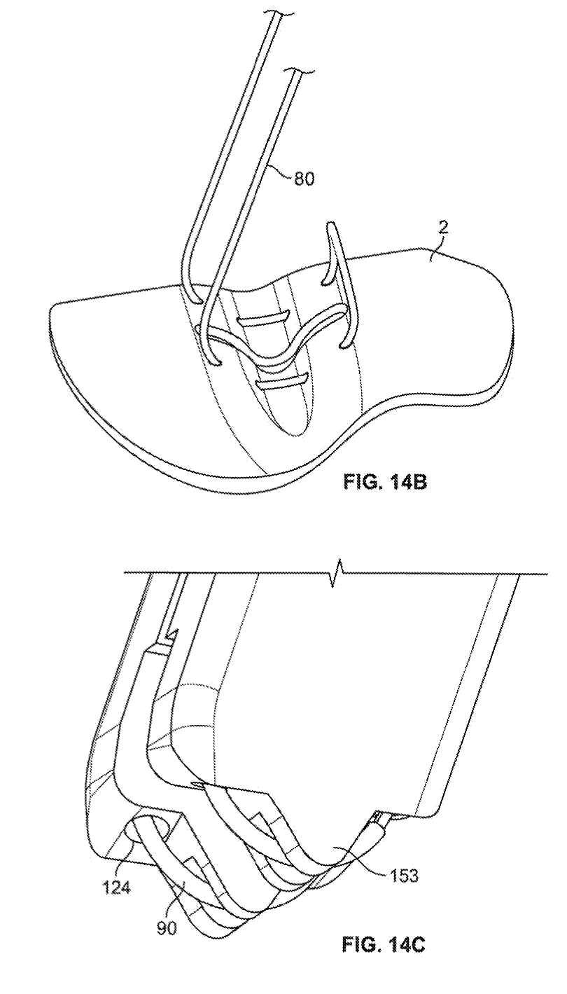

FIGS. 14A to 14C illustrate an example of a clamp assembly 140 and main body 102 having respective corresponding recesses 151 and protrusions 15 to position the clamped tissue in a defined shape. With the tissue 2 in a defined shape, the needle assembly can make multiple penetrations through the tissue. FIG. 14B shows the suture 80 placed in a purse string stitch pattern in the tissue 2. FIG. 14C, with the clamp assembly removed for clarity, shows the curved portion of the needles assemblies 90 advanced and re-entering into the needle retrieving openings 124.

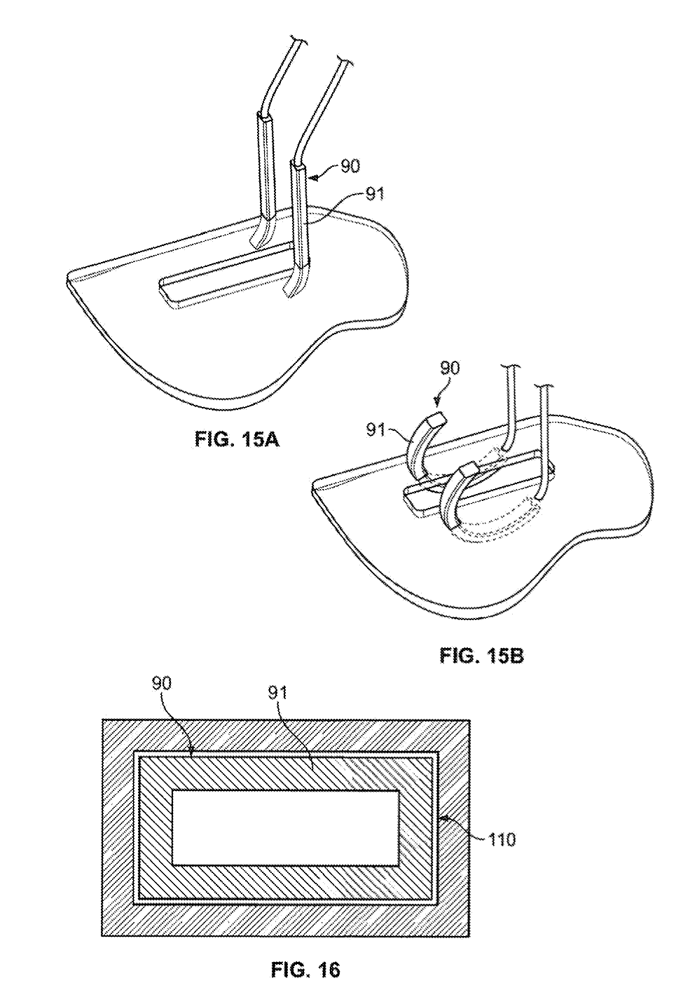

FIGS. 15A to 15B show a needle assembly 90 where the curved portion of the needle assembly or the entire needle has a non-circular cross sectional shape 91 that corresponds to the cross sectional shape of the restraining channel (not shown) to orient the needle, that it exits and travels in a defined path. The cross sectional shape could be rectangular, oval, or any polygonal shape to orient the needle, or have numerous variations that provide a feature to orient the needle assembly relative to the restraining channel. As shown in FIG. 16, the needle assembly 90 has a cross sectional shape where an outer dimension taken along a horizontal axis is larger than an outer dimension taken along a vertical axis. Accordingly; as shown in FIGS. 15A and 15B, the needle assembly 90 bends about the longer dimension or the horizontal axis. In one example, the cross sectional shape can be described as a non-circular cross sectional shape 91. In any case, the cross sectional shape of the needle assembly and the restraining channel can be sized to accommodate a sliding fit. In some variations, restraining portion/constraining channel will have a similar non-circular cross sectional shape 100 as that of the needle assembly 90.

1. A suture driving assembly for positioning a suture in a tissue section, the assembly comprising:
at least one needle assembly having a tissue piercing end distal to an elongate shaped section, the elongate shaped section having a curvilinear shape, the elongate shaped section being elastically deformable when restrained into a strained state and upon release assumes the curvilinear shape, the suture coupled to the needle assembly;
a main body having a tissue engaging surface at a distal end, at least one constraining channel and at least one retrieving channel each of which having an opening at the tissue engaging surface;
such that when the elongate shaped section of the needle assembly is in the constraining channel, the elongate shaped section is deformed into the strained state and when the elongate shaped section advances through the guide segment portion, the elongate shaped section assumes the curvilinear shape, upon continued advancement the elongated shaped section exits through the opening of constraining channel in the curvilinear shape;
a suture retriever assembly located in the needle retrieving channel;
a clamping member axially moveable relative to the tissue engaging surface, the clamping member having a first profile configured to advance through an opening in the tissue section and where the clamping member can be withdrawn toward the tissue supporting face to secure the tissue section therebetween.

2. The suture driving assembly of claim 1, where the elongate shaped section comprises a non-circular cross-sectional shape.

3. The suture driving assembly of claim 2, where the constraining channel comprises at least a portion having a non-circular cross-sectional shape to allow a sliding fit with the non-circular cross-sectional shape of the elongate section of the needle assembly.

4. The suture driving assembly of claim 1, where the clamping member comprises an expandable second profile.

5. The suture driving assembly of claim 4, where the clamping member comprises a living hinge structure.

6. The suture driving assembly of claim 1, where the clamping member comprises at least one recess and the tissue engaging surface comprises at least one protrusion, where the protrusion and recess are removably nestable within each other to cause tissue secured therebetween to assume a shape.

7. The suture driving assembly of claim 1, where the at least one needle assembly comprises at least two needle assemblies.

8.-13. (canceled)

14. The suture driving assembly of claim 1, where the needle assembly comprises a needle lumen extending through at least the tissue piercing end and where the suture is removably nested within the needle lumen.

15. The suture driving assembly of claim 14, where the suture is exterior to the needle assembly and a first free end of the suture is inserted into the needle lumen at the tissue piercing end.

16. The suture driving assembly of claim 15, where a second free end of the suture is coupled to a second needle assembly having a second tissue piercing end distal to a second elongate shaped section, where the second free end of the suture is inserted into a second needle lumen at the second tissue piercing end.

17. The suture driving assembly of claim 1, where the suture is removably front-loaded into the tissue piercing end.

18. The suture driving assembly of claim 17, where the suture retriever assembly comprises at least one pawl member that reduces an opening of the retrieving channel to less than a size of the needle assembly and suture, where the pawl member is biased to allow movement of the needle assembly and suture in a first direction and resist movement of the needle assembly and suture in a second direction, where rearward movement of the needle assembly from the retrieving channel causes the paw member to compress and retain the suture within the retrieving channel.

19.-20. (canceled)

21. The suture driving assembly of claim 1, where the shaped section comprises a plurality of curved segments such that advancement of the needle assembly causes the tissue piercing end to penetrate tissue at a plurality of locations.

22. (canceled)

23. The suture driving assembly of claim 1, where the constraining channel maintains the strained state of the shaped section in a substantially linear shape.

24.-32. (canceled)

33. A suture driving assembly for closing an opening in a tissue section, the assembly comprising:
a first needle assembly having a tissue piercing end distal and being elastically deformable when restrained into a strained state and upon release assumes the curvilinear shape;
a suture exterior to the needle assembly and having at least one end front-loaded into a needle lumen of a first tissue piercing portion of the first needle assembly;
a main body having a tissue engaging surface at a distal end, at least one constraining channel and at least one retrieving channel each of which having an opening at the tissue engaging surface;
where the constraining channel extends through the main body and comprises at least a restraining portion having a profile to maintain the needle assembly into the strained state and a guide segment portion adjacent to the constraining channel opening and having a profile to release needle assembly into the curvilinear shape when advanced therethrough and upon continued advancement the needle assembly exits the opening of the constraining channel in the curvilinear shape;
a suture retriever assembly located in the needle retrieving channel and comprising a pawl mechanism, where the pawl mechanism interferes with the front loaded suture and needle assembly when advanced therein, where rearward movement of the front loaded suture and needle assembly causes the pawl to engage the suture to retain the suture within the needle retrieving channel; and
an clamping member axially moveable relative to the tissue engaging surface, the clamping member having a first profile, where the clamping member can advance through an opening in the tissue section and where the clamping member can be withdrawn toward the tissue supporting face to secure the tissue section therebetween when expanded.

34.-39. (canceled)

40. The suture driving assembly of claim 33, further comprising a second needle assembly where a second end of the suture is front loaded into a second tissue piercing portion of the second needle assembly, where a second intermediate section of the suture extends along an exterior of the needle assembly

41.-44. (canceled)

45. The suture driving assembly of claim 33, where the pawl mechanism reduces a diameter of the retrieving channel to less than a size of the needle assembly and suture, where the pawl mechanism is biased to allow movement of the needle assembly and suture in a first direction and resist movement of the needle assembly and suture in a second direction, where rearward movement of the needle assembly from the retrieving channel causes the paw mechanism to compress and retain the suture within the retrieving channel.

46.-54. (canceled)

55. A method for positioning a suture in a wall of an organ to close an opening in the wall, the method comprising:
placing a main body adjacent to a proximal side of the tissue, where the main body comprises at least one needle assembly coupleable to the suture and within a constraining channel located in the main body, where the needle assembly comprises a tissue piercing end distal to an elongate shaped section, the elongate shape section having a curvilinear shape, the shaped section being elastically deformable into a strained state within the constraining channel, and a suture coupled to the needle assembly, the main body further including a tissue engaging surface;
advancing a clamping member through the opening in the Organ when the clamping member is in a reduced profile;
positioning the wall of the organ between the main body and the clamping member;
advancing the needle assembly from the constraining channel into a guide segment, where the guide segment permits the shaped section of the needle assembly located therein to revert to the curvilinear shape prior to leaving the guide segment and entering the wall of the organ;
driving the needle assembly through a proximal side of the wall of the organ, such that the shaped section moves through the curvilinear shape so that the tissue piercing distal end and suture re-enter the main body at a retrieving channel; and
withdrawing the main body.

56. The method of claim 55, where the elongate shape of the needle assembly comprises a non-circular cross-sectional shape, such that the non-cylindrical shape biases the elongate shape to assume the curvilinear shape

57.-91. (canceled)

92. A suture driving assembly for positioning a suture in a tissue section, the assembly comprising:
at least one needle assembly having a tissue piercing end distal to an elongate shaped section, the elongate shaped section having a curvilinear shape and a non-circular cross-sectional shape, the elongate shaped section being elastically deformable when restrained into a strained state and upon release assumes the curvilinear shape, the suture coupled to the needle assembly;
a main body having a tissue engaging surface at a distal end, at least one constraining channel and at least one retrieving channel each of which having an opening at the tissue engaging surface;
such that the constraining channel having a non-circular cross-sectional shape that corresponds to the non-circular cross-sectional shape of the respective elongate shaped section deployed therein, wherein an orientation between the constraining channel and the shaped section of the needle is provided, such that upon advancement the elongate shaped section of the needle exits the distal end of the constraining channel, the needle travels in a path defined by its curvilinear shape and the cross-sectional shape of the respective constraining channel and needle assembly;
a suture retriever assembly located in the needle retrieving channel; and
a clamping member axially moveable relative to the tissue engaging surface, the clamping member having a first profile configured to advance through an opening in the tissue section and where the clamping member can be withdrawn toward the tissue supporting face to secure the tissue section therebetween.

93. (canceled)
